(19) United States
(12) Patent Application Publication
(10) Pub. No.: US 2024/0307351 A1
(43) Pub. Date: Sep. 19, 2024

(54) AGENT FOR ANTI-AGING OR FOR CELLULAR REJUVENATION

(71) Applicant: TOHOKU UNIVERSITY, Sendai-Shi (JP)

(72) Inventors: Takaaki Abe, Miyagi (JP); Shin-ichiro Kanno, Miyagi (JP); Takehiro Suzuki, Miyagi (JP); Ko Hashimoto, Miyagi (JP); Tomoko Kasahara, Miyagi (JP); Fumika Nanto-Hara, Ibaraki (JP)

(21) Appl. No.: 18/539,931

(22) Filed: Dec. 14, 2023

(30) Foreign Application Priority Data
Dec. 26, 2022 (JP) 2022-208385

(51) Int. Cl. A61K 31/405; A61K 31/454; A61P 1/16; A61P 3/04

(52) U.S. Cl. CPC A61K 31/405; A61K 31/454; A61P 1/16; A61P 3/04

(57) ABSTRACT

It is to provide an agent for anti-aging or cellular rejuvenation, and an agent for preventing or ameliorating symptoms or diseases associated with aging, in which a low-molecular compound that can be produced relatively simply and inexpensively is contained as an active ingredient.

Provided are an agent for anti-aging or cellular rejuvenation and a preventive or ameliorative agent for a symptom or disease associated with aging. Such agents contain one or more compounds selected from the group consisting of a compound represented by General Formula (I₀), a compound represented by General Formula (II), a compound represented by General Formula (III) and pharmaceutically acceptable salts thereof when R³ is OH.

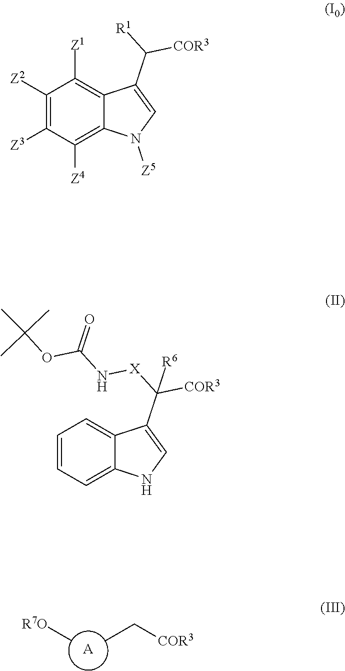

### TECHNICAL FIELD

The present invention relates to an agent for anti-aging or cellular rejuvenation, the agent comprising one or more compounds selected from the group consisting of: a compound represented by General Formula (I₀), a compound represented by General Formula (II), and a compound represented by General Formula (III), which are described later, and pharmaceutically acceptable salts thereof when R³ is OH (hereinafter, these may be collectively referred to as “the present compound group”), and a preventive or ameliorative agent for a symptom or disease associated with aging, the preventive or ameliorative agent including the agent for anti-aging or cellular rejuvenation.

### BACKGROUND ART

The studies on aging control have found that calorie restriction to an extent that does not cause undernutrition exhibits an anti-aging and life extension effect on archaea, yeast, nematodes, and humans. Sir2 has been identified as one of the molecules involved in these effects. There are seven sirtuin families as their mammalian homologs. Among them, Sirtuin 1, which is most similar in structure and function to Sir2, is attracting attention.

Sirtuin 1 has NAD-dependent deacetylase activity and ADP-ribosyl transferase activity, and plays an important role in vivo. For example, in Sirtuin-1 highly-expressing mice, amelioration in glucose metabolism, cholesterol metabolism, and fat metabolism has been observed together with amelioration in physical ability and prolongation of the reproductive period. In addition, amelioration in glucose tolerance and inhibition of fatty liver under high-fat diet have also been observed. That is, activation of Sirtuin 1 is considered to be useful for prevention, treatment, or amelioration of metabolic diseases (Non-Patent Document 1).

In addition, it has been revealed that activation of Sirtuin 1 deacetylates the p65 subunit of the transcription factor NF-κB (nuclear factor-kappa B) to attenuate the NF-κB activity, resulting in inhibition of inflammation, and this inflammation inhibition action is considered to prevent, treat, or ameliorate inflammatory diseases (Non-Patent Document 1). Further, it has been found that the proliferation capability of the skin is enhanced by blocking the gene of NF-κB in mice, and activation of Sirtuin 1 is considered to be useful for enhancing the proliferation capability of the skin (Non-Patent Document 2).

Sirtuin 1 deacetylates FOXO, p53, p73, Ku70, Smad7 and the like, and as a result, induces oxidative stress resistance and cell-death inhibition. The induction of these phenotypes is considered to contribute to the realization of anti-aging and life extension (Non-Patent Document 1).

Cellular senescence is caused by exposure to exogenous stress such as ultraviolet radiation. Cellular senescence is a phenomenon in which the cell cycle is permanently arrested. It has been revealed that Sirtuin 1 regulates the expression of telomere reverse transcriptase (TERT), maintains stable telomeres by deacetylating histones, maintains stable genomes by promoting DNA repair by deacetylating repair proteins such as Werner syndrome proteins (WRN proteins), and inhibits cellular senescence through these functions (Non-Patent Document 3).

It has been revealed like this that Sirtuin 1 has various functions such as a diabetes amelioration effect, a cardiovascular protection effect, a renal disease amelioration effect, and a neuroprotective effect, in addition to a metabolic disease amelioration effect, an inflammatory disease amelioration effect, and a cellular senescence amelioration effect. Therefore, activation of sirtuin 1 is considered to be useful for prevention, treatment, or amelioration of various diseases such as metabolic diseases, inflammatory diseases, cellular senescence, diabetes, cardiovascular diseases, renal diseases, and neurological diseases.

Resveratrol, which is abundantly contained in the skin of red grapes, is known as a material that activates Sirtuin 1. In recent years, there have been reported a Sirtuin-1 activator including a plant body of Peucedanum japonicum or a solvent extract thereof (Patent Document 1), a sirtuin activator derived from an extract of Krachai Dam (Patent Document 2), and a composition for enhancing the expression of Sirtuin 1 including an extract of Tongkat ali (Patent Document 3). Patent Document 4 discloses a method for upregulating Sirtuin 1 using an antisense oligonucleotide against a natural antisense transcript for Sirtuin 1.

On the other hand, the inventors of the present invention have reported that 4-(2,4-difluorophenyl)-2-(1H-indole-3-yl)-4-oxo-butanoic acid (i.e., MA5) has an effect of enhancing erythropoietin expression and treating mitochondrial diseases (Patent Document 5), an effect of inhibiting organ fibrosis (Patent Document 6), and an effect of preventing or ameliorating hearing loss (Patent Document 7). However, it has not been known that the present compound group has a SIRT protein production promoting effect, an anti-aging effect, a cellular rejuvenating effect and the like.

### CITATION LIST

### Patent Document

Patent Document 1: JP 5666053 B

Patent Document 2: JP 2018-199680 A

Patent Document 3: JP 2021-075501 A

Patent Document 4: JP 2017-221221 A

Patent Document 5: WO 2014/080640 A

Patent Document 6: JP 2015-189670 A

Patent Document 7: JP 2019-116453 A

### Non-Patent Document

Non-Patent Document 1: Chemistry and Biology, 2009, Vol. 47, No. 8, pp. 531-537

Non-Patent Document 2: Aging cell, 2010, 9, pp. 285-290

Non-Patent Document 3: BMB Reports, 2019, 52 (1), pp. 24-34

### SUMMARY OF INVENTION

### Technical Problem

An object of the present invention is to provide an agent for anti-aging or cellular rejuvenation, or a preventive or ameliorative agent for preventing or ameliorating symptoms or diseases associated with aging, in which a low-molecular compound that can be produced relatively simply and inexpensively is contained as an active ingredient.

### Solution to Problem

The inventors of the present invention have been conducting intensive studies to solve the above-described issues. In the process, the inventors have found:
(a) the present compound group described later has an action of effectively promoting the production of SIRT proteins (for example, an action of effectively increasing the amount of a SIRT protein in a cell);
(b) the present compound group described later has an effect of ameliorating fatty liver and obesity, which are symptoms or diseases associated with aging, and an effect of ameliorating liver function;
(c) the present compound group described later has an action of ameliorating decline in motor function associated with aging;
(d) the present compound group described later has actions such as an increase in the expression of FOXO1 Gene, activation of IGF1 signaling pathway, inhibition of NF-kB pathway with PPAR Gene, activation of longevity regulating pathway, and an increase in the expression of a group of genes related to pluripotency; and
(e) the present compound group described later has an effect of inhibiting methylation of DNA in a pathway associated with aging;
and the like, and
they have completed the present invention.

It is known that an increase in the expression of FOXO1 Gene, activation of the IGF1 signaling pathway, inhibition of the NF-kB pathway, activation of the longevity regulating pathway, and an increase in the expression of a group of genes related to pluripotency are associated with longevity, anti-aging, and the like.

That is, the present invention is as follows.

[1] An agent for anti-aging or cellular rejuvenation, the agent comprising one or more compounds selected from the group consisting of:
a compound represented by General Formula (I0):

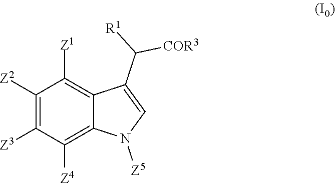

wherein
   R¹ represents a hydrogen atom; a benzoylmethyl group in which a benzene ring is unsubstituted or substituted with an alkyl group having 1 to 7 carbon atoms, an alkoxyl group having 1 to 7 carbon atoms, and fluorine and/or chlorine; a chain or branched alkyl group having 4 to 6 carbon atoms, the alkyl group being unsubstituted or substituted with fluorine; or methylene or ethylene substituted with a phenyl group, a cyclopentyl group, or a piperidinyl group, wherein the phenyl group may be further substituted with one or more phenyl groups, and N of the piperidinyl group may be further substituted with an acyl group,
   Z¹, Z², Z³, and Z⁴ may be identical or different, and represent a hydrogen atom, a halogen atom, a C1 to C6 alkyl group, a C2 to C6 alkenyl group, a C2 to C6 alkynyl group, or an organic oxy group represented by OR⁸, wherein R⁸ represents a C1 to C7 alkyl group, a C2 to C6 alkenyl group, a C2 to C6 alkynyl group, or a benzyl group,
   Z⁵ represents a hydrogen atom or a C1 to C6 alkyl group, and
   R³ is a group selected from the group consisting of OH, OR⁴, NHR⁴, and NR⁴R⁵, wherein R⁴ and R⁵ are identical or different and are a substituted or unsubstituted alkyl group having 1 to 4 carbon atoms;
a compound represented by General Formula (II):

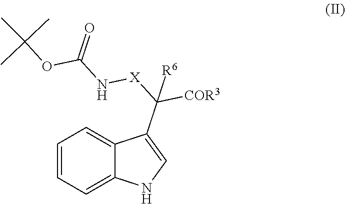

wherein
   R⁶ is a hydrogen atom or a methyl group,
   X is an alkylene group having 4 to 6 carbon atoms or an ether group having 4 carbon atoms, and
   R³ is a group selected from the group consisting of OH, OR⁴, NHR⁴, and NR⁴R⁵, wherein R⁴ and R⁵ are identical or different and are a substituted or unsubstituted alkyl group having 1 to 4 carbon atoms;
a compound represented by General Formula (III):

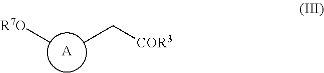

wherein
   A represents indole or naphthalene,
      wherein
         when A is indole, an acetic acid group and R⁷O are substituted at a third position and a fifth position of indole, respectively, and
         when A is naphthalene, an acetic acid group and R⁷⁰ are substituted at a first position and a seventh position of naphthalene, respectively,
   R⁷ represents an alkyl group having 1 to 5 carbon atoms or a benzyl group, wherein a benzene ring of the benzyl group may be substituted with 1 or 2 or more alkyl groups having 1 to 3 carbon atoms or alkoxy groups having 1 to 3 carbon atoms,
   R³ is a group selected from the group consisting of OH, OR⁴, NHR⁴, and NR⁴R⁵, wherein R⁴ and R⁵ are identical or different and are a substituted or
unsubstituted alkyl group having 1 to 4 carbon atoms; and pharmaceutically acceptable salts thereof when R³ is OH.

[2] The agent for anti-aging or cellular rejuvenation according to [1], wherein an SIRT protein includes one or more proteins selected from SIRT1 protein, SIRT2 protein, SIRT3 protein, SIRT4 protein, SIRT5 protein, SIRT6 protein, and SIRT7 protein.

[3] The agent for anti-aging or cellular rejuvenation according to [1] or [2], wherein the compound is a compound represented by the following Formula (I-1), Formula (II-1), Formula (I-2), Formula (I-3), Formula (I-4), Formula (I-5) or Formula (III-1), or a pharmaceutically acceptable salt thereof:

Formula (I-1) (Compound #5 described later in Examples):

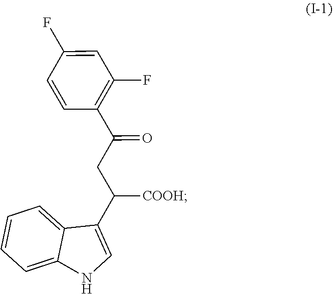 [SMILES: O=C(CC(C(=O)O)c1c[nH]c2ccccc12)c1ccc(F)cc1F]

Formula (II-1) (Compound #13 described later in Examples):

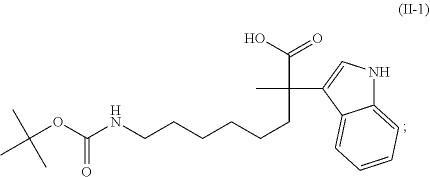 [SMILES: CC(C)(C)OC(=O)NCCCCCCC(C)(C(=O)O)c1c[nH]c2ccccc12]

Formula (I-2) (Compound #9 described later in Examples):

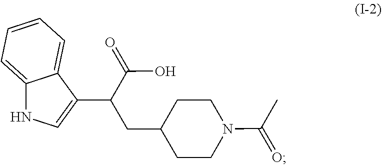 [SMILES: CC(=O)N1CCC(CC(C(=O)O)c2c[nH]c3ccccc23)CC1]

Formula (I-3) (Compound #21 described later in Examples):

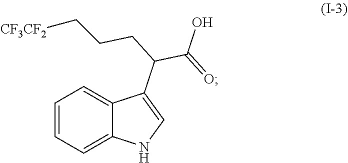

Formula (I-4) (Compound #30 described later in Examples):

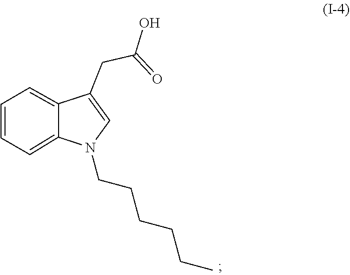 [SMILES: CCCCCCn1cc(CC(=O)O)c2ccccc21]

Formula (I-5) (Compound #4 described later in Examples):

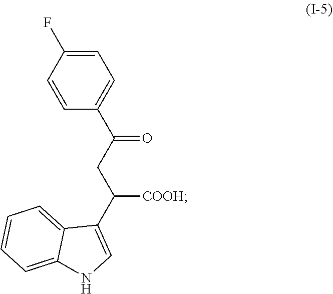 [SMILES: O=C(CC(C(=O)O)c1c[nH]c2ccccc12)c1ccc(F)cc1]

Formula (III-1) (Compound #35 described later in Examples):

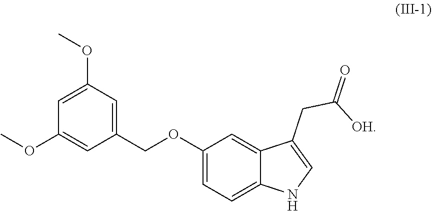 [SMILES: COc1cc(COc2ccc3[nH]cc(CC(=O)O)c3c2)cc(OC)c1]

[4] A preventive or ameliorative agent for a symptom or disease associated with aging, the preventive or ameliorative agent comprising the agent for anti-aging or cellular rejuvenation described in any one of [1] to [3].

[5] The preventive or ameliorative agent according to [4], wherein the symptom or disease associated with aging is one or more selected from the group consisting of obesity, liver disease, decline in liver function, renal fibrosis, renal dysfunction, and decline in motor function.

Examples of another embodiment of the present invention include: a method for anti-aging or rejuvenating cells in an animal in need of anti-aging or cellular rejuvenation, the method comprising administering one or more compounds selected from the present compound group to the animal; one or more compounds selected from the present compound group for use as an agent for anti-aging or cellular rejuvenation; one or more compounds selected from the present compound group for use in anti-aging or cellular rejuvenation; and use of one or more compounds selected from the present compound group for producing an agent for anti-aging or cellular rejuvenation.

Examples of another embodiment of the present invention include: a method for preventing or ameliorating (treating) a symptom or disease associated with aging (preferably caused by aging), the method comprising administering one or more compounds selected from the present compound group to a subject in need of prevention or amelioration (treatment) of a symptom or disease associated with aging (preferably caused by aging); one or more compounds selected from the present compound group for use as a preventive or ameliorative (therapeutic) agent for a symptom or disease associated with aging (preferably caused by aging); one or more compounds selected from the present compound group for use in preventing or ameliorating (treating) a symptom or disease associated with aging (preferably caused by aging); and use of one or more compounds selected from the present compound group for producing a preventive or ameliorative (therapeutic) agent for a symptom or disease associated with aging (preferably caused by aging).

### Advantageous Effects of Invention

According to the present invention, it is possible to provide an agent for anti-aging or cellular rejuvenation, and a preventive or ameliorative agent for symptoms or diseases associated with aging. Further, the present invention adopts a low-molecular compound that can be produced relatively simply and in a high yield as an active ingredient for anti-aging or cellular rejuvenation, and thus enables relatively simple and inexpensive production.

### BRIEF DESCRIPTION OF DRAWINGS

FIG. 1-1 is a diagram showing the results of analyzing the expression levels of various SIRTs (SIRT1, isoform 1 of SIRT2, isoform 2 of SIRT2, and SIRT3) in cells obtained by culturing a mouse inner ear cell line (HEI-OC1 cell line) under the presence of 0.1% DMSO or MA5 at various concentrations (1 μM, 3 μM, 10 μM, or 30 μM) by Western blotting. The lower graphs show the results in which each band intensity in the above results is expressed as a relative value when the band intensity in 0.1% DMSO as a control is set to 1.

FIG. 1-2 is a diagram showing the results of analyzing the expression levels of various SIRTs (SIRT5, SIRT6, and SIRT7) and the expression level of B-actin in cells of a HEI-OC1 cell line cultured in the presence of 0.1% DMSO or MA5 at various concentrations (1 μM, 3 μM, 10 μM, or 30 μM) by Western blotting. The lower graphs show the results in which each band intensity in the above results is expressed as a relative value when the band intensity in 0.1% DMSO as a control is set to 1.

FIG. 2 is a diagram showing the effect on brown adipocytes in brown adipose tissue when MA5 (Compound #5) is administered to NDUFS4 (NADH: ubiquinone oxidoreductase subunit S4) homozygous knockout mice (Ndufs4−/−mice). The upper panel shows the results of HE (hematoxylin-eosin) staining of brown adipose tissue of mice when Compound #5 is administered to NDUFS4 homozygous knockout mice (Ndufs4−/−mice) at a dose of 10 mg/day per kg of mouse weight for 50 days [Compound #5 administration group]. The lower panel shows the results of HE (hematoxylin-eosin) staining of brown adipose tissue of mice when CMC (carboxymethylcellulose) is administered to NDUFS4 homozygous knockout mice (Ndufs4−/−mice) at a dose of 10 mg/day per kg of mouse weight for 50 days [CMC administration group; Control group].

FIG. 3 is a diagram showing the results of quantitative PCR analysis of the expression of UCP1 (uncoupling protein 1) Gene, PPARγ (peroxisome proliferator-activated receptor gamma) Gene, and FABP4 (fatty acid binding protein 4) Gene in brown adipocytes of Ndufs4−/−mice in [Compound #5 administration group] and [Control group] shown in FIG. 2. AU on the vertical axis of the graph represents an expression level ratio when the expression level of Control group is set to 1.

FIG. 4 is a diagram showing the effect on fatty liver when MA5 (Compound #5) is orally administered to NDUFS4 (NADH: ubiquinone oxidoreductase subunit S4) homozygous knockout mice (Ndufs4−/−mice). The left panels of the figure are electron microscopic images of liver tissue in a group of wild-type mice to which water is administered, the middle panels are electron microscopic images of liver tissue in a group of Ndufs4−/−mice to which water is administered, and the right panels are electron microscope images of liver tissue in a group of Ndufs4−/−mice to which Compound #5 is administered at a dose of 0.1 kg/day per mg of mouse weight for 24 days (Compound #5 administration group). “X2000” and “X10000” on the left side of the figure represent magnifications.

FIG. 5 is a diagram showing the effect on liver function when Compound #4 is orally administered to C57BL6 mice (8 weeks old). “Epo” represents erythropoietin concentration (IU/L) in blood of the mice, “TG” represents triglyceride concentration (mg/dL) in blood of the mice, “GOT” represents glutamic oxaloacetic transaminase concentration (IU/L) in blood of the mice, “GPT” represents glutamic-pyruvate transaminase concentration (IU/L) in blood of the mice, “T-Cho” represents total cholesterol concentration (mg/dL) in blood of the mice, and “HDL” represents HDL cholesterol concentration (mg/dL) in blood of the mice. In each panel, “DMSO” is the result of Control group (n=4), “L” is the result of the group (n=4) to which Compound #4 is administered at a dose of 50 mg/day per kg of mouse weight for 7 days, and “H” is the result of the group (n=4) to which Compound #4 is administered at a dose of 150 mg/day per kg of mouse weight for 7 days.

FIG. 6 is a diagram showing the results of detecting the amount of SIRTI protein in RH30 cells by Western blotting. From the left of the figure, the results of [Compound #5 addition group] to which MA5 (Compound #5) was added, [Compound #9 addition group] to which Compound #9 was added, [Compound #13 addition group] to which Compound #13 was added, [Compound #21 addition group] to which Compound #21 was added, [Compound #30 addition group] to which Compound #30 was added, [Compound #35 addition group] to which Compound #35 was added, and [Control group] (DMSO) as a control are shown. Compounds #5, #9, #13, #21, #30 and #35 are all the present compounds. As a loading control, the results of β-actin are shown at the bottom.

FIG. 7 is a diagram showing the results of quantification of each band in the detection results of the Western blotting in FIG. 6 through normalization with the band of β-actin as a loading control. From the left of the figure, the results of [Control group] (DMSO) as a control, [Compound #5 addition group] to which MA5 (Compound #5) was added, [Compound #9 addition group] to which Compound #9 was added, [Compound #13 addition group] to which Compound #13 was added, [Compound #21 addition group] to which Compound #21 was added, [Compound #30 addition group] to which Compound #30 was added, and [Compound #35 addition group] to which Compound #35 was added are shown.

FIG. 8 includes graphs showing the results of measuring the running time on a treadmill of aged mice in [MA5 addition group] (right panel) and [Vehicle group] (left panel). The vertical axis represents the running time (seconds) on the treadmill, and the horizontal axis represents the number of weeks elapsed from the start of measurement. In the figure, ** represents p<0.01.

FIG. 9 is a diagram showing the percentage of survival of the aged mice in [MA5 addition group] and [Vehicle group] (percentage of mice that continue running without being exhausted until the elapsed time) (%). The result with a lower percentage of survival represents the result of [Vehicle group], and the result with a higher percentage of survival represents the result of [MA5 addition group]. The horizontal axis represents the elapsed time (minutes) from the start of the test, and the vertical axis (Probability of Survival) represents the percentage (%) of mice that continue running without being exhausted.

FIG. 10 is a diagram showing the results of RNA sequencing (RNA-Seq) analysis performed on the muscle cells of aged mice in [MA5 addition group] and [Vehicle group] to identify genes whose expression levels were changed (increased or decreased) in [MA5 addition group] and [Vehicle group] compared to the muscle cells of young mice before administration. In FIG. 10, the results in the five columns on the right are the results of [MA5 addition group], and the results in the five columns on the left are the results of [Vehicle group].

FIG. 11 is a diagram showing the results of comparison of the expression levels of Klf4 Gene, Pou5f1 (Oct4) Gene, Sox2 Gene, and Myc Gene, which are pluripotency-related genes, between muscle cells of aged mice in [Vehicle group] and muscle cells of aged mice in [MA5 addition group]. The result of comparison of the expression level of Nanog Gene is also shown. In the figure, * represents p<0.05, and ** represents p<0.01.

FIG. 12 is a diagram showing the results of pathway analysis of genes whose expression has varied in muscle cells of aged mice in [MA5 addition group]. The first from the top is muscle structure development, the sixth from the top is longevity regulating pathway-multiple species, the tenth from the top is negative regulation of MAPK cascade, and the second from the bottom is insulin signaling.

FIG. 13 is a diagram showing the relationship between genes whose expression has varied in muscle cells of aged mice in [MA5 addition group] and transcription factors that control the expression of the genes or upstream genes of the genes. The genes listed on the left side of the figure represent genes whose expression levels increased or decreased in [MA5 addition group] as compared with [Vehicle group]. Genes regulated downstream of the transcription factors listed above in the figure are marked immediately below the name of the transcription factor. For example, genes regulated downstream of FOXO1 include ITGA5, RCANI, and the like.

FIG. 14 is a diagram showing the results of measuring the running time on a treadmill of young mice in [Vehicle group], [MA5 low dose addition group], and [MA5 high dose addition group]. The horizontal axis represents the number of weeks elapsed from the start of the test, and the vertical axis represents the relative running time when the running time on the treadmill before the start of the test (0 week) is set to 1. In the figure, represents the result of [MA5 high dose addition group], A represents the result of [MA5 low dose addition group], and o represents the result of [Vehicle group].

FIG. 15 is a diagram showing the percentage of survival (percentage of mice that continue running without being exhausted until the elapsed time) (%) of young mice in [Vehicle group], [MA5 low dose addition group] (Low dose), and [MA5 high dose addition group] (High dose). The result with the highest percentage of survival represents the result of [MA5 high dose addition group], the result with the second highest percentage of survival represents the result of [MA5 low dose addition group], and the result with the lowest percentage of survival represents the result of [Vehicle group]. The horizontal axis represents the elapsed time (minutes) from the start of the test, and the vertical axis (Probability of Survival) represents the percentage (%) of mice that continue running without being exhausted.

FIG. 16 is a diagram showing the results of RNA sequencing (RNA-Seq) analysis performed on the muscle cells of young mice in [MA5 addition group] and [Vehicle group] to identify genes whose expression levels changed (increased or decreased) in [MA5 addition group] and [Vehicle group] compared to the muscle cells of young mice before administration. In FIG. 16, the results in the five columns on the right are the results of [MA5 addition group], and the results in the five columns on the left are the results of [Vehicle group].

FIG. 17 is a diagram showing the results of pathway analysis of genes whose expression has varied in muscle cells of young mice in [MA5 addition group].

FIG. 18 is a diagram showing the relationship between genes whose expression has varied in muscle cells of young mice in [MA5 addition group] and transcription factors that control the expression of the genes or upstream genes of the genes. The genes listed on the left side of the figure represent genes whose expression levels increased or decreased in [MA5 addition group] as compared with [Vehicle group]. Genes regulated downstream of the transcription factors listed above in the figure are marked immediately below the name of the transcription factor. For example, genes regulated downstream of SUZ¹² include SMYD2, MPZ, and the like.

FIG. 19 is a diagram showing the relationship between genes whose expression has varied in liver cells of young mice in [MA5 addition group] and transcription factors that control the expression of the genes or upstream genes of the genes. The genes listed on the left side of the figure represent genes whose expression levels increased or decreased in [MA5 addition group] as compared with [Vehicle group]. Genes regulated downstream of the transcription factors listed above in the figure are marked immediately below the name of the transcription factor. For example, genes regulated downstream of MYC include GSTT2, SLC6A6, and the like.

FIG. 20 is a diagram showing the relationship between genes whose expression has varied in liver cells of aged mice in [MA5 addition group] and transcription factors that control the expression of the genes or upstream genes of the genes. The genes listed on the left side of the figure represent genes whose expression levels increased or decreased in [MA5 addition group] as compared to those before administration. Genes regulated downstream of the transcription factors listed above in the figure are marked immediately below the name of the transcription factor. For example, genes regulated downstream of FOXO1 include BCL6, CYP17A1, and the like.

FIG. 21 is a diagram showing the results of pathway analysis of genes whose expression has varied in cells of the cochlea of the inner ears of Ndufs4 knockout mice in [MA5 addition group]. The fifth from the top is a pathway related to the regulation of insulin like growth factor transport and uptake.

FIG. 22 is a diagram showing the results of evaluating the survival rates of two types of groups (Klotho homo KO mouse non-administered group [“Vehicle” in the figure] and Klotho homo KO mouse administered group [“MA-5” in the figure]) with survival curves based on the Kaplan-Meier method.

FIG. 23 is a diagram showing the results of relative quantification of mRNA expression levels of two types of fibrosis-related genes (aSMA [a-actin microfilament] and Collal [type I collagen fibers]) in renal tissue of three types of groups (wild-type untreated group [“Wild-type” in the figure], Klotho homo KO mouse non-administrated group [“α-Klotho KO Vehicle” in the figure], and Klotho homo KO mouse administrated group [“α-Klotho KO MA-5” in the figure]) by a real-time PCR method.

FIG. 24 is a diagram showing the results of measuring the amount of spermidine in the plasma of three types of groups (wild-type non-administered group [“Wild-type” in the figure], Klotho homo KO mouse non-administered group [“KO dw” in the figure], and Klotho homo KO mouse administered group [“KO #5” in the figure]).

### DESCRIPTION OF EMBODIMENTS

An agent for anti-aging or cellular rejuvenation of the present invention (which hereinafter may be referred to as “the present agent”) can be used only “for anti-aging” or “for cellular rejuvenation”, and contains, as an active ingredient, one or more compounds selected from the present compound group. A preventive or ameliorative agent for a symptom or disease associated with aging (which hereinafter may be referred to as “the present preventive/ameliorative agent”) can be used only “for preventing or ameliorating a symptom or disease associated with aging”, and contains the present agent. The present agent and the present preventive/ameliorative agent may use the present compound group as their active ingredient in such a manner that the present compound group is used alone as a food or drink product or a pharmaceutical product (preparation), or that the present compound group is used as a composition form (food and drink product composition or pharmaceutical composition) by further mixing additives. Examples of such food and drink product include health foods (functional foods, nutritional supplements, dietary supplements, nutrient-enriched foods, nutrient-controlled foods, supplements, and the like) and health-promoting food products (foods for specified health use, foods with nutrient function claims, foods with functional claims, and the like).

The present compound group can effectively promote the production of an SIRT protein. In the present specification, the “SIRT protein” include one or more selected from the group consisting of SIRT1, SIRT2, SIRT3, SIRT4, SIRT5, SIRT6, and SIRT7 in an animal (preferably human).

In the present specification, a “SIRT protein” may be a variant when the variant exists. The amino acid sequence of each SIRT protein of humans is disclosed in the following accession numbers: The amino acid sequence of each SIRT protein of humans and other organisms can be found on websites such as GenBank (trade name).
Human SIRT1 isoform a; NP_036370.2
Human SIRTI isoform b; NP_001135970.1
Human SIRTI isoform c; NP 001300978.1
Human SIRT2 isoform 1; NP_036369.2
Human SIRT2 isoform 2; NP 085096.1
Human SIRT2 isoform 3; NP_001180215.1
Human SIRT3 mitochondrial isoform a; NP 036371.1
Human SIRT3 mitochondrial isoform b; NP_001017524.1
Human SIRT3 mitochondrial isoform c; NP_001357239.1
Human SIRT3 mitochondrial isoform d; NP_001357241.1
Human SIRT3 mitochondrial isoform e; NP 001357243.1
Human SIRT3 mitochondrial isoform f; NP 001357244.1
Human SIRT3 mitochondrial isoform g; NP_001357245.1
Human SIRT3 mitochondrial isoform h; NP_001357246.1
Human SIRT3 mitochondrial isoform i; NP_001357250.1
Human SIRT3 mitochondrial isoform j; NP_001357253.1
Human SIRT4 mitochondrial isoform 1; NP_001372662.1
Human SIRT4 mitochondrial isoform 2; NP 001372663.1
Human SIRT5 mitochondrial isoform 1; NP_001363727.1
Human SIRT5 mitochondrial isoform 2; NP 001363737.1
Human SIRT5 mitochondrial isoform 3; NP_001180196.1
Human SIRT5 mitochondrial isoform 4; NP_001229756.1
Human SIRT5 mitochondrial isoform 5; NP 001363738.1
Human SIRT5 mitochondrial isoform 6; NP_001363740.1
Human SIRT5 mitochondrial isoform 7; NP 001363742.1
Human SIRT5 mitochondrial isoform 8; NP 001363744.1
Human SIRT6 isoform 1; NP_057623.2
Human SIRT6 isoform 2; NP 001180214.1
Human SIRT6 isoform 3; NP 001307987.1
Human SIRT6 isoform 4; NP_001307988.1
Human SIRT6 isoform 5; NP_001307989.1
Human SIRT6 isoform 6; NP_001307990.1
Human SIRT6 isoform 7; NP_001307991.1
Human SIRT6 isoform 8; NP 001307992.1
Human SIRT6 isoform 9; NP_001307993.1
Human SIRT7; NP 057622.1

A detailed description of the compounds included in the present compound group is provided below.

In one aspect of the present invention, R¹ in General Formula (I₀) is a hydrogen atom; or a benzoylmethyl group in which a benzene ring is unsubstituted or substituted with an alkyl group having 1 to 7 carbon atoms, an alkoxyl group having 1 to 7 carbon atoms, fluorine and/or chlorine. The benzene ring of the benzoylmethyl group may be substituted, and examples of the substituted group include: a benzoylmethyl group having 1 to 5 alkyl groups having 1 to 7 carbon atoms, 1 to 5 alkoxyl groups having 1 to 7 carbon atoms, 1 to 5 fluorine atoms, 1 to 5 chlorine atoms on the benzene ring; or a benzoylmethyl group having alkyl groups having 1 to 4 carbon atoms, alkoxyl groups having 1 to 4 carbon atoms, 1 to 5 fluorine atoms and chlorine atoms in total on the benzene ring. Here, the alkyl group having 1 to 7 carbon atoms may be a linear or branched alkyl group, and examples thereof include a methyl group, an ethyl group, a propyl group, a 2-ethylbutyl group, a 2-methylpentyl group, and a 3-methylpentyl group.

Preferred examples include a methyl group, a 2-ethylbutyl group, a 2-methylpentyl group, or a 3-methylpentyl group.

The alkoxyl group having 1 to 7 carbon atoms may be a linear or branched alkoxyl group, and examples thereof include a methoxy group, an ethoxy group, and a propoxy group.

In another aspect of the present invention, R¹ in General Formula (I₀) is a chain or branched alkyl group having 4 to 6 carbon atoms that is unsubstituted or substituted with fluorine. Examples of the chain or branched alkyl group having 4 to 6 carbon atoms that is unsubstituted or substituted with fluorine include an n-butyl group, an isobutyl group, a sec-butyl group, an n-pentyl group, a 2-methylpentyl group, a 3-methylpentyl group, a 2-ethylbutyl group, and fluorinated compounds thereof. Preferred examples include a 4,4,5,5,5-pentafluoropentyl group, a 2-ethylbutyl group, a 2-methylpentyl group, a 3-methylpentyl group, a 4,4,5,5,5-pentafluoropentyl group, or a 4,4,5,5,5-pentafluoropentyl group.

In another aspect of the present invention, R₁ in General Formula (I₀) is methylene or ethylene substituted with a phenyl group, a cyclopentyl group, or a piperidinyl group. The phenyl group may be further substituted with one or more phenyl groups. N of the piperidinyl group may be further substituted with an acyl group. Examples of the acyl group include an acetyl group and a benzoyl group. The methylene or ethylene substituted with a phenyl group, a cyclopentyl group, or a piperidinyl group is a benzyl group, a 2-phenethyl group, a cyclopentylmethyl group, or a 2-cyclopentylethyl group. Examples of the benzyl group or 2-phenethyl group substituted with one or more phenyl groups include a 3-phenylbenzyl group, a 2-(1, 1′-biphenyl-3-yl)-ethyl group, a 2-(1,l′-biphenyl-4-yl)-ethyl group, and a 2-(3,5-diphenylphenyl)-ethyl group. Preferred examples of R¹ in General Formula (I) include a 2-phenethyl group, a 2-cyclopentylethyl group, a 2-(1,1′-biphenyl-3-yl)-ethyl group, and a 1-acetyl-4-piperidinylmethyl group.

Z¹, Z², Z³, and Z⁴ in General Formula (I₀) may be identical or different and represent a hydrogen atom, a halogen atom, C1 to C6 alkyl, C2 to C6 alkenyl, C2 to C6 alkinyl, or an organic oxy group represented by OR⁸, wherein R⁸ represents C1 to C7 alkyl, C2 to C6 alkenyl, C2 to C6 alkinyl, or benzyl group. Z⁵ may be a hydrogen atom or C1 to C7 alkyl. Examples of the halogen atom include a fluorine atom, a chlorine atom, a bromine atom, and an iodine atom. C to C7 alkyl may be a linear or branched alkyl group, and examples thereof include a methyl group, an ethyl group, a propyl group, an isopropyl group, an n-butyl group, an isobutyl group, an n-hexyl group, and an n-heptyl group. Preferred examples include a methyl group, an ethyl group, a propyl group, an n-butyl group, an n-hexyl group, and an n-heptyl group. C2 to C6 alkenyl may be a linear or branched alkenyl group, and examples thereof include an ethenyl group (vinyl group), a 1-propenyl group, and a 2-propenyl group (allyl group). C1 to C7 alkoxyl group (when R⁸ is C1 to C7 alkyl in the organic oxy group represented by OR⁸) may be a linear or branched alkoxyl group, and examples thereof include a methoxy group, an ethoxy group, a propoxy group, an isopropoxy group, an n-butoxy group, an isobutoxy group, a sec-butoxy group, an n-pentoxy group, and an n-heptyloxy group. Preferred examples include a methoxy group, an ethoxy group, a propoxy group, an n-butoxy group, an n-pentoxy group, and an n-heptyloxy group. Preferably, Z¹, Z², Z³, and Z⁴ may be identical or different and are hydrogen, methoxy, fluorine, or chlorine.

In General Formula (I₀), R³ is a group selected from the group consisting of OH, OR⁴, NHR⁴, and NR⁴R⁵, wherein R⁴ and R⁵ are identical or different and are a substituted or unsubstituted alkyl group having 1 to 4 carbon atoms. Examples of the substituted or unsubstituted alkyl group having 1 to 4 carbon atoms include a methyl group, an ethyl group, a propyl group, an isopropyl group, an n-butyl group, an isobutyl group, a sec-butyl group, a tert-butyl group, pyrrolidine in which R⁴ and R⁵ are combined with nitrogen, and those substituted with a methoxy group, a phenyl group, fluorine, and chlorine. Preferred examples include a methyl group, a monochloromethyl group, an ethyl group, a 2-methoxyethyl group, a 2,2,2-trichloroethyl group, a 1-phenylethyl group, a 2-phenylethyl group, a methoxyethyl group, an isopropyl group, a hexafluoroisopropyl group, and pyrrolidine, and more preferred examples include a methyl group and an ethyl group.

An example of the embodiment of General Formula (I₀) includes a compound represented by the following General Formula (I). A preferred example includes a compound represented by General Formula (1).

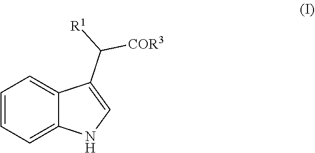

wherein R¹ and R³ have the same meanings as defined in [1] above.

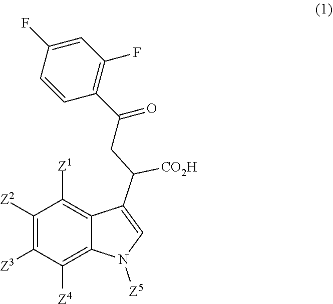

wherein Z¹, Z², Z³, Z⁴, and Z⁵ have the same meanings as defined in [1] above.

Z¹, Z², Z³, and Z⁴ in Formula (1) are preferably a hydrogen atom, a halogen atom, C1 to C6 alkyl, or an organic oxy group represented by OR⁸, more preferably a hydrogen atom, a fluorine atom, a chlorine atom, C1 to C3 alkyl such as a methyl group, an ethyl group, an n-propyl group, or an isopropyl group, or an organic oxy group represented by OR^{8.}

Z⁵ is preferably a hydrogen atom or C1 to C3 alkyl, more preferably a hydrogen atom or a methyl group.

R⁸ is preferably C1 to C6 alkyl, and more preferably C1 to C3 alkyl such as a methyl group, an ethyl group, an n-propyl group, or an isopropyl group, or a benzyl group.

Among the compounds represented by Formula (1), compounds in which any one substituent of Z¹, Z², Z³, Z⁴, or Z⁵ is substituted is preferable.

In the above Formulas (2), (3), (4), (5), and (6), Z¹, Z², Z³, Z⁴, and Z⁵ have the same definitions as Z¹, Z², Z³, Z⁴, and Z⁵ in Formula (1).

Specific examples of the compound represented by Formula (1) include the following compounds.

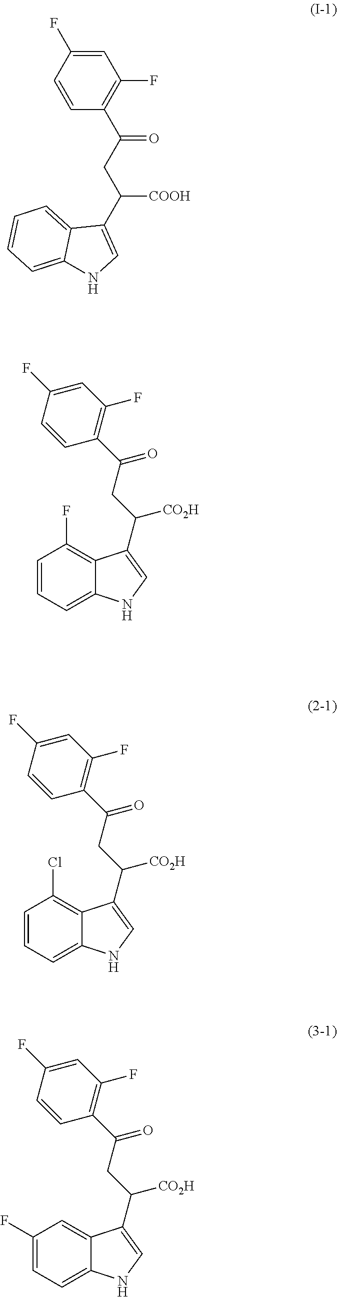 [SMILES: O=C(CC(C(=O)O)c1c[nH]c2ccccc12)c1ccc(F)cc1F ; O=C(CC(C(=O)O)c1c[nH]c2cccc(F)c12)c1ccc(F)cc1F ; O=C(CC(C(=O)O)c1c[nH]c2cccc(Cl)c12)c1ccc(F)cc1F ; O=C(CC(C(=O)O)c1c[nH]c2ccc(F)cc12)c1ccc(F)cc1F]

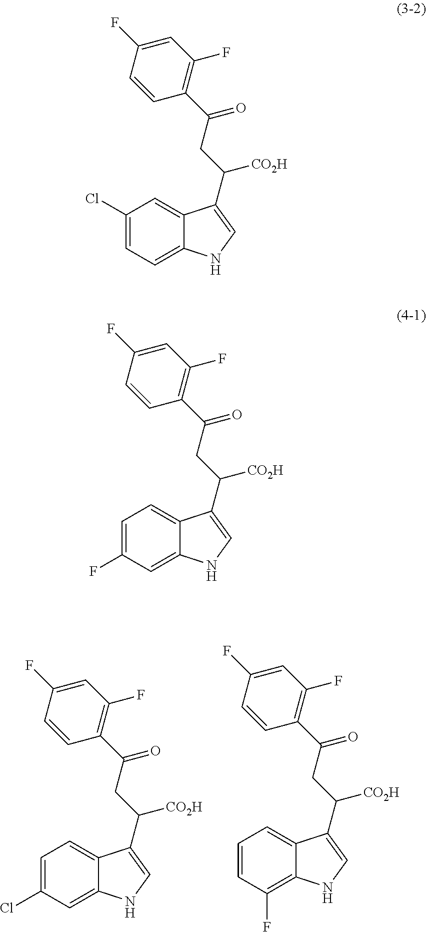 [SMILES: O=C(CC(C(=O)O)c1c[nH]c2ccc(Cl)cc12)c1ccc(F)cc1F ; O=C(CC(C(=O)O)c1c[nH]c2cc(F)ccc12)c1ccc(F)cc1F ; O=C(CC(C(=O)O)c1c[nH]c2cc(Cl)ccc12)c1ccc(F)cc1F ; O=C(CC(C(=O)O)c1c[nH]c2c(F)cccc12)c1ccc(F)cc1F]

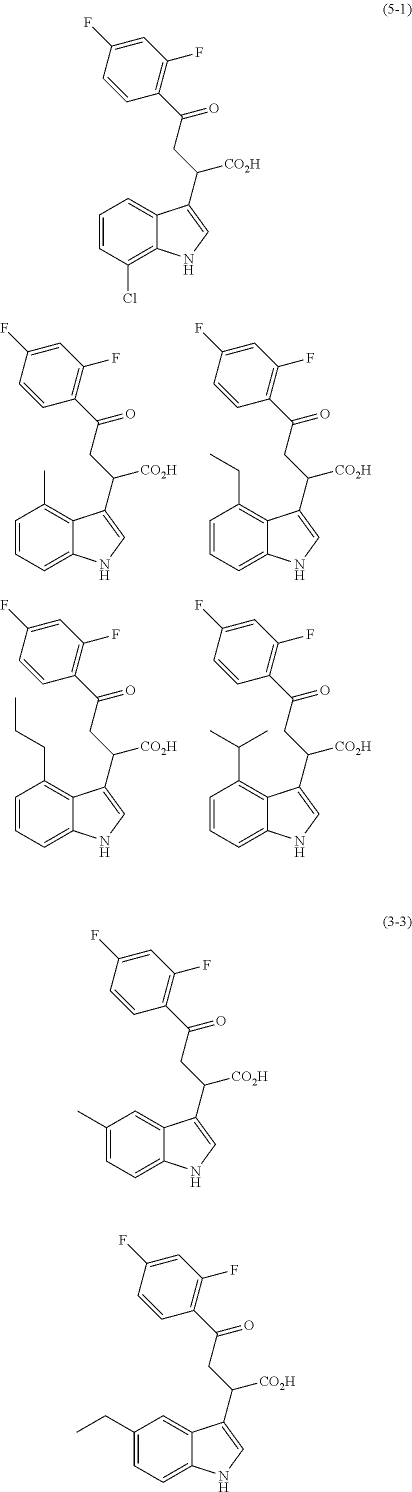

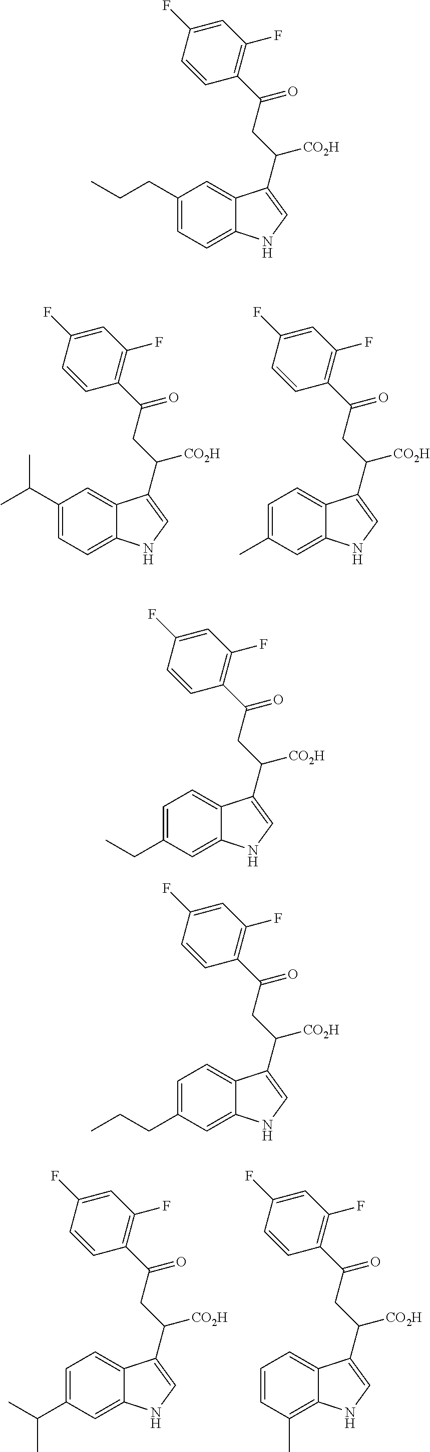

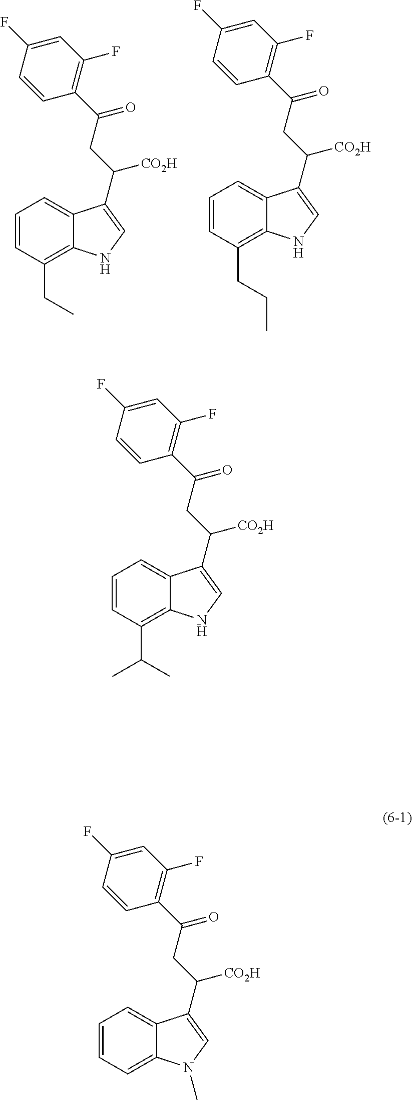 [SMILES: CCc1cccc2c(C(CC(=O)c3ccc(F)cc3F)C(=O)O)c[nH]c12 ; CCCc1cccc2c(C(CC(=O)c3ccc(F)cc3F)C(=O)O)c[nH]c12 ; CC(C)c1cccc2c(C(CC(=O)c3ccc(F)cc3F)C(=O)O)c[nH]c12 ; Cn1cc(C(CC(=O)c2ccc(F)cc2F)C(=O)O)c2ccccc21]

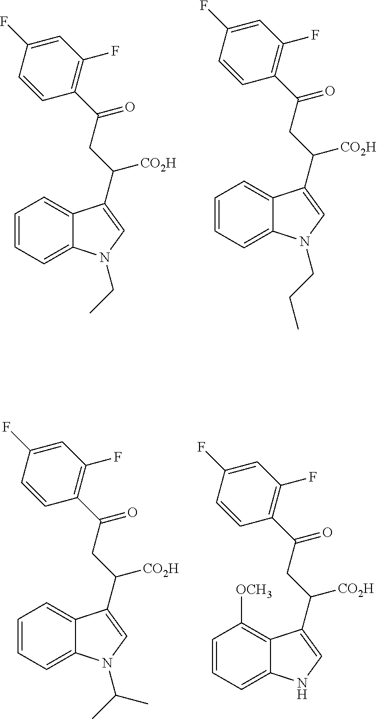 [SMILES: CCn1cc(C(CC(=O)c2ccc(F)cc2F)C(=O)O)c2ccccc21 ; CCCn1cc(C(CC(=O)c2ccc(F)cc2F)C(=O)O)c2ccccc21 ; CC(C)n1cc(C(CC(=O)c2ccc(F)cc2F)C(=O)O)c2ccccc21 ; COc1cccc2[nH]cc(C(CC(=O)c3ccc(F)cc3F)C(=O)O)c12]

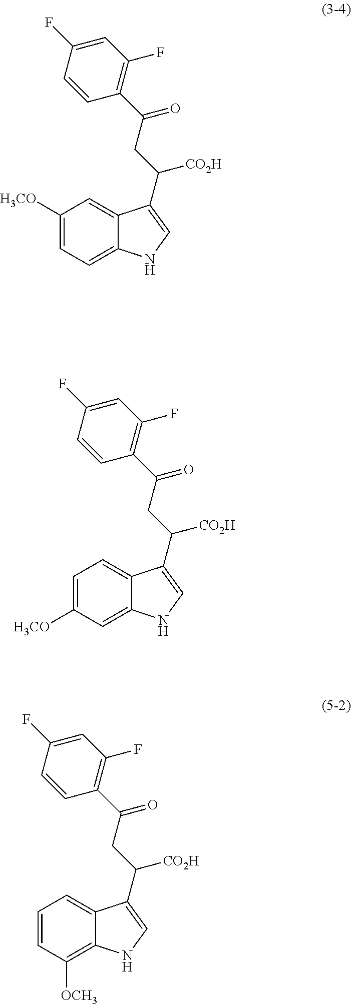 [SMILES: COc1ccc2[nH]cc(C(CC(=O)c3ccc(F)cc3F)C(=O)O)c2c1 ; COc1ccc2c(C(CC(=O)c3ccc(F)cc3F)C(=O)O)c[nH]c2c1 ; COc1cccc2c(C(CC(=O)c3ccc(F)cc3F)C(=O)O)c[nH]c12]

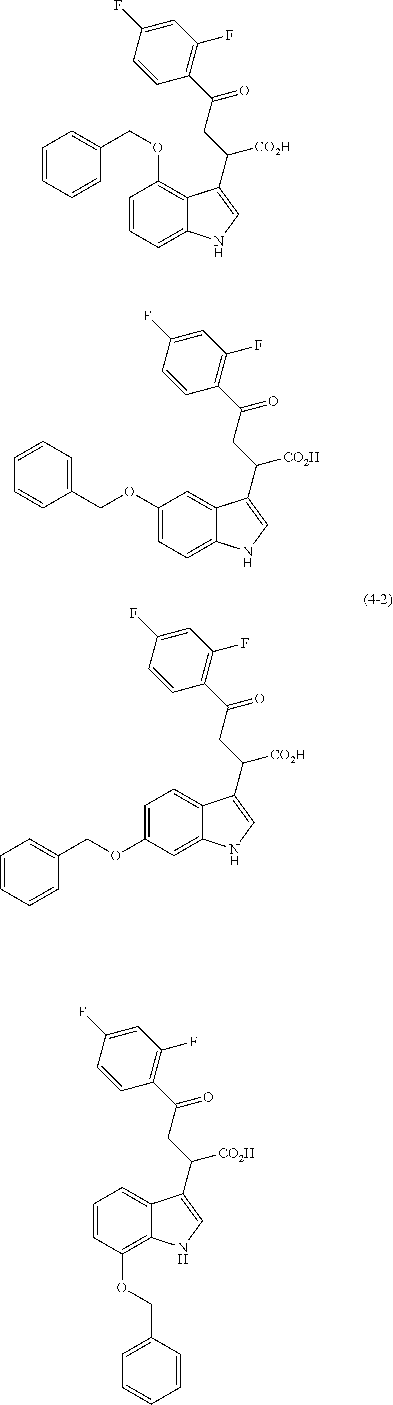 [SMILES: O=C(CC(C(=O)O)c1c[nH]c2cccc(OCc3ccccc3)c12)c1ccc(F)cc1F ; O=C(CC(C(=O)O)c1c[nH]c2ccc(OCc3ccccc3)cc12)c1ccc(F)cc1F ; O=C(CC(C(=O)O)c1c[nH]c2cc(OCc3ccccc3)ccc12)c1ccc(F)cc1F ; O=C(CC(C(=O)O)c1c[nH]c2c(OCc3ccccc3)cccc12)c1ccc(F)cc1F]

Among the above compounds, the compound of (1-1), (2-1), (3-1), (3-2), (3-3), (3-4), (4-1), (4-2), (5-1), (5-2), or (6-1) is preferable.

When R₁ in General Formula (I₀) is a 2,4-difluorobenzoylmethyl group, Z¹, Z², Z³, Z⁴, and Z⁵ are hydrogen, and R³ is OH, the compound represented by General Formula (I₀) represents Compound #5 (also referred to as “MA5”) described later in Examples. When R₁ in General Formula (I₀) is a 4-fluorobenzoylmethyl group, Z¹, Z², Z³, Z⁴, and Z⁵ are hydrogen, and R³ is OH, the compound represented by General Formula (I₀) represents Compound #4 described later in Examples. When R₁ in General Formula (I₀) is a 1-acetyl-4-piperidinylmethyl group, Z¹, Z², Z³, Z⁴, and Z⁵ are hydrogen, and R³ is OH, the compound represented by General Formula (I₀) represents Compound #9 described later in Examples. When R¹ in General Formula (I₀) is a 4,4,5,5,5-pentafluoropentyl group, Z¹, Z², Z³, Z⁴, and Z⁵ are hydrogen, and R³ is OH, the compound represented by General Formula (I₀) represents Compound #21 described later in Examples. When R¹ in General Formula (I₀) is hydrogen, Z¹, Z², Z³, and Z⁴ are hydrogen, Z⁵ is a hexyl group, and R³ is OH, the compound represented by General Formula (I₀) represents Compound #30 described later in Examples.

X in General Formula (II) is a linear alkylene group having 4 to 6 carbon atoms, that is, butylene-(CH₂)₄—, pentylene-(CH₂)₅—, hexylene-(CH₂)₆—, or an ether group having 4 carbon atoms. Examples of the ether group having 4 carbon atoms include a methylene-O-propylene group, an ethylene-O-ethylene group, and a propylene-O-methylene group. A butylene group, a hexylene group, and an ethylene-O-ethylene group are preferable.

R⁴ and R⁵ in General Formula (II) are identical or different and represent a substituted or unsubstituted alkyl group having 1 to 4 carbon atoms. Examples of the substituted or unsubstituted alkyl group having 1 to 4 carbon atoms include a methyl group, an ethyl group, a propyl group, an isopropyl group, an n-butyl group, an isobutyl group, a sec-butyl group, a tert-butyl group, pyrrolidine in which R⁴ and R⁵ are combined with nitrogen, and those substituted with a methoxy group, a phenyl group, fluorine, and chlorine. Preferred examples include a methyl group, a monochloromethyl group, an ethyl group, a 2,2,2-trichloromethyl group, a 1-phenylethyl group, a 2-phenylethyl group, a methoxyethyl group, an isopropyl group, a hexafluoroisopropyl group, and pyrrolidine, and more preferred examples include a methyl group and an ethyl group.

As a specific example of the compounds represented by General Formula (II), Compound #13 described later in Examples is a compound in which X in General Formula (II) is a hexylene group, R⁶ is a methyl group, and R³ is OH.

R⁷ in General Formula (III) is an alkyl group having 1 to 5 carbon atoms or a benzyl group. Examples of the chain or branched alkyl group having 1 to 5 carbon atoms include a methyl group, an ethyl group, a propyl group, an isopropyl group, an n-butyl group, an isobutyl group, a sec-butyl group, a tert-butyl group, an n-pentyl group, a 1-methylbutyl group, a 2-methylbutyl group, a 3-methylbutyl group, a 1-ethylpropyl group, a 1, 1-dimethylpropyl group, a 1,2-dimethylpropyl group, and a 2,2-dimethylpropyl group. The benzene ring of the benzyl group may be substituted with one or more alkyl groups having 1 to 3 carbon atoms or alkoxy groups having 1 to 3 carbon atoms. Examples of the alkyl group having 1 to 3 carbon atoms include a methyl group, an ethyl group, an n-propyl group, and an isopropyl group. Examples of the alkoxy group having 1 to 3 carbon atoms include a methoxy group, an ethoxy group, an n-propoxy group, and an isopropoxy group. R⁷ in General Formula (III) is preferably a methyl group, an ethyl group, a propyl group, an n-butyl group, an n-pentyl group, or a 3,5-dimethoxybenzyl group, and more preferably a 3,5-dimethoxybenzyl group.

In General Formula (III), R⁴ and R⁵ are identical or different and are a substituted or unsubstituted alkyl group having 1 to 4 carbon atoms. Examples of the substituted or unsubstituted alkyl group having 1 to 4 carbon atoms include a methyl group, an ethyl group, a propyl group, an isopropyl group, an n-butyl group, an isobutyl group, a sec-butyl group, a tert-butyl group, pyrrolidine in which R⁴ and R⁵ are combined with nitrogen, and those substituted with a methoxy group, a phenyl group, fluorine, and chlorine. Preferred examples include a methyl group, a monochloromethyl group, an ethyl group, a 2,2,2-trichloromethyl group, a 1-phenylethyl group, a 2-phenylethyl group, a methoxyethyl group, an isopropyl group, a hexafluoroisopropyl group, and pyrrolidine, and more preferred examples include a methyl group and an ethyl group.

When A in General Formula (III) is indole, R⁷ is a 3,5-dimethoxybenzyl group, and R³ is OH, the compound represented by General Formula (III) represents Compound #35 described later in Examples.

When a compound selected from the present compound group has an asymmetric carbon atom and an asymmetric point related to an axial chirality, the compound includes all possible optical isomers, and the optical isomers can be used in any ratio. For example, an optically active compound can be used in the form of an enantiomer, a racemate, or a mixture of enantiomers in any ratio, and when there is a plurality of asymmetric points, the optically active compound may be used in the form of a mixture of diastereomers in any ratio.

Pharmaceutically acceptable salts in the present compound group include metallic salts made from aluminum, calcium, lithium, magnesium, potassium, sodium, and zinc, and organic salts made from N,N′-dibenzylethylenediamine, chloroprocaine, choline, diethanolamine, ethylenediamine, N-methylglucamine, lysine, procaine, and the like.

As a method for synthesizing a compound selected from the present compound group, methods disclosed in Patent Document 5, WO 2019/235455 A, and the like can be used, but the method is not limited to these methods, and a commonly known synthesis method can be used. The compounds shown below are available from Sigma-Aldrich Co. LLC, Tokyo Chemical Industry Co., Ltd., Wako Pure Chemical Industries, Ltd., Kanto Chemical Co., Ltd., and the like. With respect to a reaction solvent and a reaction temperature, unless otherwise specified, the reaction is performed in a solvent and at a temperature usually used for the reaction. The reaction is usually performed under an argon or nitrogen atmosphere. Protecting groups can be used with reference to Green & Wuts, “PROTECTIVE GROUPS in ORGANIC SYNTHESIS” 3^{rd} ed. John Wiley & Sons, Inc.

Preferable examples of the present compound group include Compounds #5, #4, #9, #13, #21, #30, and #35 whose effects are specifically shown in Examples of the present specification.

Examples of additives of the present agent and the present preventive/ameliorative agent include typical pharmaceutically acceptable compounding ingredients such as carriers, binders, stabilizers, excipients, diluents, pH buffers, disintegrators, isotonic agents, additives, coating agents, solubilizers, lubricants, glidants, solubilizing agents, anti-adherents, flavoring agents, sweeteners, solvents, gelling agents, and nutrients. Specific examples of such compounding ingredients include water, physiological saline, animal fats and oils, vegetable oils, lactose, starch, gelatin, crystalline cellulose, gum, talc, magnesium stearate, hydroxypropyl cellulose, polyalkylene glycol, polyvinyl alcohol, and glycerin.

Examples of the administration form of the present agent and the present preventive/ameliorative agent include oral administration in the form of powders, granules, tablets, capsules, syrups, suspensions, and the like, and parenteral administration in the form of solutions, emulsions, suspensions, and the like through injection or intranasal administration in the form of sprays.

The dose of the present agent and the present preventive/ameliorative agent is appropriately determined depending on age, body weight, sex, symptom, sensitivity to drugs, and the like. It is usually administered in a dosage range of 1 μg to 200 mg/day, preferably in a dosage range of 2 μg to 2000 μg/day, more preferably in a dosage range of 3 to 200 μg/day, and still more preferably in a dosage range of 4 to 20 μg/day in a single dose or in divided doses (for example, 2 to 4 doses) per day, but the dosage may be adjusted depending on the situation of amelioration of symptoms.

As specifically shown in Examples described later, the present compound group has an action of effectively promoting the production of SIRT proteins, an action of ameliorating fatty liver and obesity that are symptoms or diseases associated with aging, an action of ameliorating liver function, an action of ameliorating decline in motor function associated with aging, and the like. As specifically shown in Examples described later, the present compound group also has actions such as an increase in the expression of FOXO1 Gene, activation of IGF1 signal pathway, inhibition and DNA repair of NF-kB pathway with PPAR Gene, activation of longevity regulating pathway, and an increase in the expression of a group of genes related to pluripotency. As specifically shown in Examples described later, the present compound group also has an action of inhibiting methylation of DNA that is a pathway associated with aging. From these facts, by ingesting the present compound group, it is sufficiently expected to inhibit or delay the progression of symptoms or diseases associated with aging (preferably caused by aging) or to cause cellular rejuvenation in a subject who has ingested the present compound group.

As used herein, the term “anti-aging” means inhibiting or delaying the progression of any one symptom or any one disease among symptoms or diseases associated with aging (preferably caused by aging). In the present specification, examples of the “symptom or disease associated with aging (preferably cause by aging)” include cancers (for example, breast cancer, colorectal cancer, chronic lymphocytic leukemia, chronic myelogenous leukemia, and prostate cancer); sarcomas (osteosarcoma, such as chordoma, chondrosarcoma, and Ewing's sarcoma; and soft tissue sarcoma, such as malignant peripheral nerve sheath tumor, rhabdomyosarcoma, synovial sarcoma, angiosarcoma, extraosseous Ewing's sarcoma, gastrointestinal stromal tumor, liposarcoma, fibrosarcoma, myxofibrosarcoma, leiomyosarcoma, alveolar soft tissue sarcoma, undifferentiated pleomorphic sarcoma, clear cell sarcoma, dermatofibrosarcoma protuberans, and epithelioid sarcoma); cystic fibrosis; neurodegenerative diseases or disorders (for example, Alzheimer's disease, Huntington's disease, Parkinson's disease, amyotrophic lateral sclerosis, multiple sclerosis and disorders caused by aggregation of polyglutamine); skeletal muscle diseases (for example, Duchenne muscular dystrophy, skeletal muscle atrophy, Becker muscular dystrophy or myotonic dystrophy); decline in motor function (for example, muscle weakness in skeletal muscle, sarcopenia, osteoporosis, and osteoarthritis); metabolic diseases (for example, insulin resistance, diabetes, obesity, impaired glucose tolerance, high blood cholesterol, hyperglycemia, dyslipidemia and hyperlipidemia, hypothalamus, pituitary, adrenal hormone lowering/hyperactivity, for example, primary aldosteronism); adult-onset diabetes mellitus, diabetic nephropathy, neuropathy (for example, sensory neuropathy, autonomic neuropathy, motor neuropathy, and retinopathy); bone diseases (for example, osteoporosis), blood diseases (for example, leukemia); liver diseases (for example, fatty liver, chronic hepatitis B, chronic hepatitis C, liver cirrhosis, liver cancer, alcohol-related liver disease, primary biliary cirrhosis, auto-immune hepatitis, and non-alcoholic steatohepatitis (NASH); and acute liver diseases such as acute viral hepatitis and drug-induced hepatopathy); obesity; bone resorption, age-related macular degeneration, AIDS-related dementia, infections, pulmonary fibrosis, ALS (amyotrophic lateral sclerosis), Fagile X syndrome, multiple sclerosis, Bell's palsy, atherosclerosis, heart diseases (for example, abnormal cardiac rhythm, chronic congestive heart failure, ischemic stroke, coronary artery disease and cardiomyopathy, abnormal cardiac rhythm), chronic degenerative disease (for example, heart muscle disease), kidney fibrosis, renal dysfunction (for example, acute renal failure, chronic renal failure; nephritis, such as acute glomerulonephritis, chronic glomerulonephritis, minimal change nephritis, nephrosclerosis, membranoproliferative nephritis, mesangial proliferative glomerulonephritis, membranoproliferative glomerulonephritis, crescentic glomerulonephritis, rapidly progressive glomerulonephritis, membranous glomerulonephritis, tubulointerstitial nephritis, acute pyelonephritis, chronic pyelonephritis, endocapillary proliferative nephritis, and lupus nephritis; amyloid kidney, membranous nephropathy, focal glomerulosclerosis, IgA nephropathy, acute tubular necrosis, nephrosis syndrome, diabetic nephropathy, gouty kidney, nephredema, renal tumor, renal ischemic disorder, and renal ischemia/reperfusion injury), type 2 diabetes, ulcers, cataracts, presbyopia, Guillain-Barr syndrome, hemorrhagic stroke, rheumatoid arthritis, inflammatory bowel disease, systemic lupus erythematosus (SLE), psoriasis, atopic dermatitis, osteoarthritis, osteoporosis, chronic obstructive pulmonary disease (COPD), pneumonia, skin aging, androgenic alopecia, urinary incontinence, hearing loss, visual impairment, and other conditions or symptoms characterized by death, aging, or undesired cell loss of nerve cells. When the present agent is administered to these subjects, the life span can be prolonged or the above-described diseases and symptoms can be prevented or treated by enhancing the expression of genes related to longevity, anti-aging, and the like. The present agent and the present preventive/ameliorative agent can be suitably used for the prevention or treatment of one or more selected from the group consisting of obesity, liver disease, decline in liver function, renal fibrosis, renal dysfunction, and decline in motor function (e.g., muscle strength) among the above-described diseases and symptoms. Preferred examples of such liver diseases include chronic liver diseases such as fatty liver, chronic hepatitis B, chronic hepatitis C, liver cirrhosis, liver cancer, alcohol-related liver disease, primary biliary cirrhosis, auto-immune hepatitis, and non-alcoholic steatohepatitis (NASH); and acute liver diseases such as acute viral hepatitis and drug-induced hepatopathy. Among them, fatty liver is more preferably exemplified.

The present agent is also an agent for cellular rejuvenation. In the present specification, “cellular rejuvenation” preferably includes occurrence of one or more selected from the group consisting of an increase in expression of FOXO1 Gene, activation of IGF1 signaling pathway, inhibition of NF-kB pathway, activation of longevity regulating pathway, methylation inhibition, an increase in expression of a group of genes related to pluripotency, activation of polycomb repressive complex-2 (PRC2) pathway, and an increase in spermidine concentration in blood.

Examples of the “group of genes related to pluripotency” include one or more genes selected from the group consisting of Myc Gene, Oct4 Gene, Sox2 Gene, Nanog Gene, and Oct1 Gene. The above-described “activation of IGF1 signaling pathway” means, for example, activation of a signaling pathway formed of one or more selected from the group consisting of IGF-1, PI3K, AKT, and mTOR. The above-described “inhibition of NF-k B pathway” means, for example, activation of a signaling pathway formed of one or more selected from the group consisting of NF-kB, TNF-α, LPS (lipopolysaccharides), CD40L, BAFF (B cell activating factor), RANKL (receptor activator of NF-kB ligand), LTβ (lyphoxin β), IL-1, IL-2, IL-6, IL-8, IL-12, COX2, VCAM (vascular cell adhesion molecule) and ICAM (intercellular adhesion molecule). The above-described “activation of longevity regulating pathway” means, for example, activation of a signaling pathway formed of one or more selected from the group consisting of IGF-1, PI3K, AKT, mTOR, FOXO, SOD2, CAT (catalase), ATG5, and SIRT1. The activation of the PRC2 pathway means, for example, activation of a signaling pathway formed of one or more selected from the group consisting of SuZ¹², Eed, Ezh1, Ezh2, and Rbbp4.

Examples of the organism species to which the present agent and the present preventive/ameliorative agent are administered include animals, and examples of such animals include mammals (humans or non-human mammals), birds, reptiles, amphibians, fish, and invertebrates, and among them, humans are preferable. In addition, other aspects of animals include humans and domestic animals. Here, the term “domestic animals” refers to animals raised and bred by humans. Examples of such domestic animals include non-human mammals (e.g., rodents such as mice, rats, hamsters, and guinea pigs; lagomorphs such as rabbits; ungulatas such as pigs, cows, goats, horses, and sheep; order carnivore such as dogs and cats), birds (e.g., chickens, quail, turkeys, pigeons, ducks, and geese), fish (e.g., carp and goldfish), and invertebrates (e.g., silkworms and bees).

The present agent and the present preventive/ameliorative agent may contain one or more selected from the group consisting of a SIRT protein production promoting component, an anti-aging component, and a cellular rejuvenation component other than the present compound group. However, since the present compound group alone exerts one or more selected from the group consisting of an excellent SIRT protein production promoting effect, an excellent anti-aging effect, and an excellent cellular rejuvenation effect, the present agent and the present preventive/ameliorative agent preferably do not contain one or more selected from the group consisting of a SIRT protein production promoting component (for example, the plant body of Peucedanum japonicum or the solvent extract thereof described in Patent Document 1; the extract of Krachai Dam described in Patent Document 2; the extract of Tongkat ali described in Patent Document 3; etc.), an anti-aging component, and a cellular rejuvenation component other than the present compound group.

The present invention will be described more specifically with reference to the following Examples, but the present invention is not limited by Examples. In the following Examples, chondrocytes were cultured in a 25 cm² flask under conditions of 5% CO₂/20% O₂ and 37° C. A chondrocyte culture solution used was prepared by adding 10% foetal calf serum (FCS) (manufactured by Life Technologies Corporation, REF: 26140-079), 1% insulin transfellin seline (manufactured by Life Technologies Corporation, REF: 41400-045), and 100 U/mL penicillin-100 μg/mL streptomycin mixture (manufactured by LONZA, REF: 17-602E) to 5 mL of α-MEN (a-modified eagle medium) (1×) (manufactured by Life Technologies Corporation, REF: 12571-063).

### EXAMPLES

### 1. Confirmation of SIRT Protein Production Promoting Effect of Compound #5 as Present Compound

### [Method]

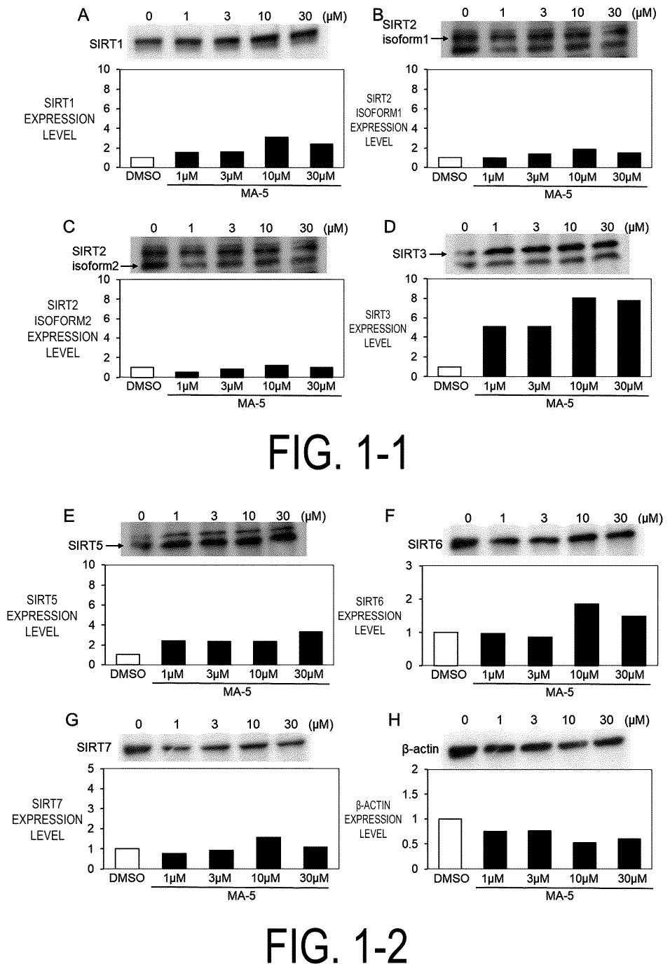

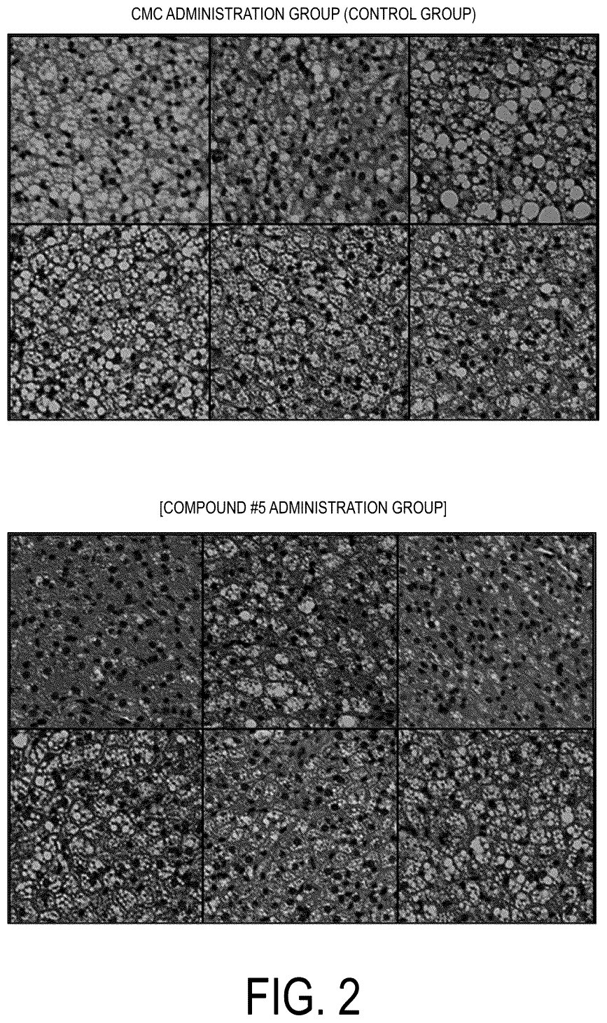

HEI-OC1 cells (mouse inner ear cell line) at 1×10⁶ cell/dish were seeded and cultured in a dish containing 10 mL of a culture solution (low-glucose-containing DMEM medium (glucose concentration of 1.0 g/dL) to which 10 wt. % of FBS was added). After one day, 0.1% DMSO as a solvent of the compound or Compound #5 at various concentrations (1 μM, 3 μM, 10 μM, or 30 μM) was added, and the mixture was cultured for 24 hours ([Compound #5 addition group]). After completion of culture, the cells were collected, disrupted by a typical method, and the expression levels of various SIRTs (SIRT1, SIRT2, SIRT3, SIRT5, SIRT6, and SIRT7) in the cells were analyzed by Western blotting (see FIGS. 1-1 and 1-2).

### [Results]

As can be seen from FIGS. 1-1 and 1-2, when the HEI-OC1 cell line was cultured under the presence of MA5, the expression levels of various SIRTs in the cells increased as compared with the case where the cell line was cultured under the absence of MA5.

This result indicates that the present compound group (Compound #5) has an effect of promoting the production of SIRT proteins in cells.

### 2. Confirmation of Obesity Ameliorating Effect of Compound #5 as Present Compound

### [Method]

Compound #5 was administered to NDUFS4 homozygous knockout mice (Ndufs4−/−mice) (28 days old) at a dose of 10 mg/day per kg of mouse weight for 50 days ([Compound #5 administration group]).

In addition, CMC (carboxymethylcellulose) was administered to NDUFS4 homozygous knockout mice (Ndufs4−/−mice) at a dose of 10 mg/day per kg of mouse weight for 50 days ([CMC administration group; Control group]).

Since the NDUFS4 homozygous knockout mice are mice with abnormal mitochondrial function and the abnormal mitochondrial function is one of aging phenomena, the NDUFS4 homozygous knockout mice can be used as one type of aging model mice.

FIG. 2 shows the results of HE (hematoxylin-eosin) staining of the brown adipose tissue of the mice in [Compound #5 administration group] and [CMC administration group].

### [Results]

In [Compound #5 administration group], a decrease in the size of lipid droplets was observed as compared with [CMC administration group] (FIG. 2). This result indicates that the present compound group (Compound #5) has an effect of ameliorating obesity. This suggests that the present compound group (Compound #5) has an ameliorating effect on obesity caused by aging.

### 3. Confirmation of Effect of Compound #5 as Present Compound on Expression of UCP1 Gene, PPARγ Gene, and FABP4 Gene

### [Method]

Compound #5 was administered to NDUFS4 homozygous knockout mice (Ndufs4−/−mice) (4 weeks old) at a dose of 10 mg/day per kg of mouse weight for 50 days ([Compound #5 administration group]).

In addition, CMC (carboxymethylcellulose) was administered to NDUFS4 homozygous knockout mice (Ndufs4−/−mice) at a dose of 10 mg/day per kg of mouse weight for 50 days ([CMC administration group; Control group]).

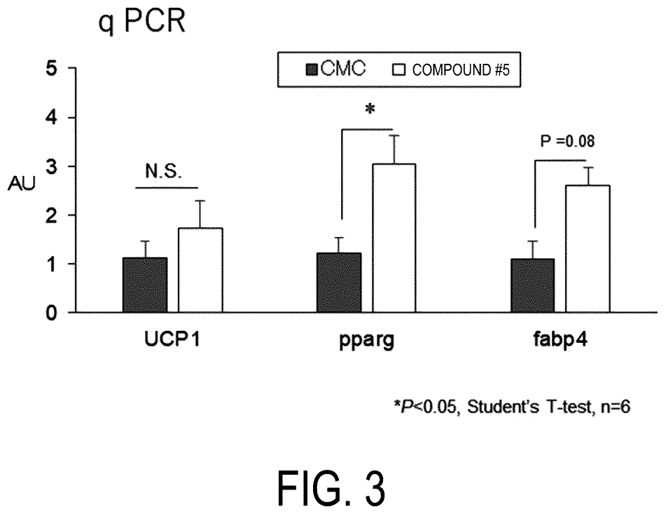

Brown adipocytes were collected from the mice of [Compound #5 administration group] and [CMC administration group]. Total RNA was extracted from the brown adipocytes, and cDNA was prepared therefrom and used for measurement of mRNA expression level. The measurement was performed by a quantitative PCR method. The expression level of mRNA was determined with a relative ratio to β-actin used as an internal standard. The results are shown in FIG. 3. As primers and probes, the following commercially available products (manufactured by Thermo Fisher Scientific Inc., and the like) were used.
Mouse Acts (actin beta, β-actin) Mm02619580_g1, Cat #4331182
Mouse Ucp1 (uncoupling protein 1), Mm00494069_m1, Cat #4331182
Mouse Pparg (PPARγ, peroxisome proliferator activated receptor gamma), Mm00440945_m1, Cat #4331182p
Mouse Fabp4 (fatty acid binding protein 4), Mm00445880_m1, Cat #4331182

### [Results]

It was shown that the expression of PPARγ Gene was significantly higher in [Compound #5 administration group] than in [CMC administration group] (FIG. 3). It was also shown that the expression of FABP4 Gene was higher in [Compound #5 administration group] than in [CMC administration group] (FIG. 3). Although the mechanism of amelioration of obesity and fatty liver in [Compound #5 administration group] (see Test 4 described below) is not clear in detail, an increase in the expression of PPARγ Gene and FABP4 Gene seems to be involved in some way. In addition, as shown in the test results described below (Tests 9. and 12. to 14. and the like described below), an anti-aging-related gene, a longevity-related gene, a reprogramming-related gene, and the like seem to be involved in the amelioration of obesity and fatty liver with Compound #5.

### 4. Confirmation of Fatty Liver Ameliorating Effect of Compound #5 as Present Compound

### [Method]

Compound #5 was orally administered to NDUFS4 homozygous knockout mice (Ndufs4−/−mice) (4 weeks old) at a dose of 0.1 kg/day per mg of mouse weight for 24 days ([Compound #5 administration group]).

In addition, water was orally administered to NDUFS4 homozygous knockout mice (Ndufs4−/−mice) for 24 days ([DMSO group; Control 1 group]). Further, water was orally administered to wild-type mice for 24 days ([Control 2 group]).

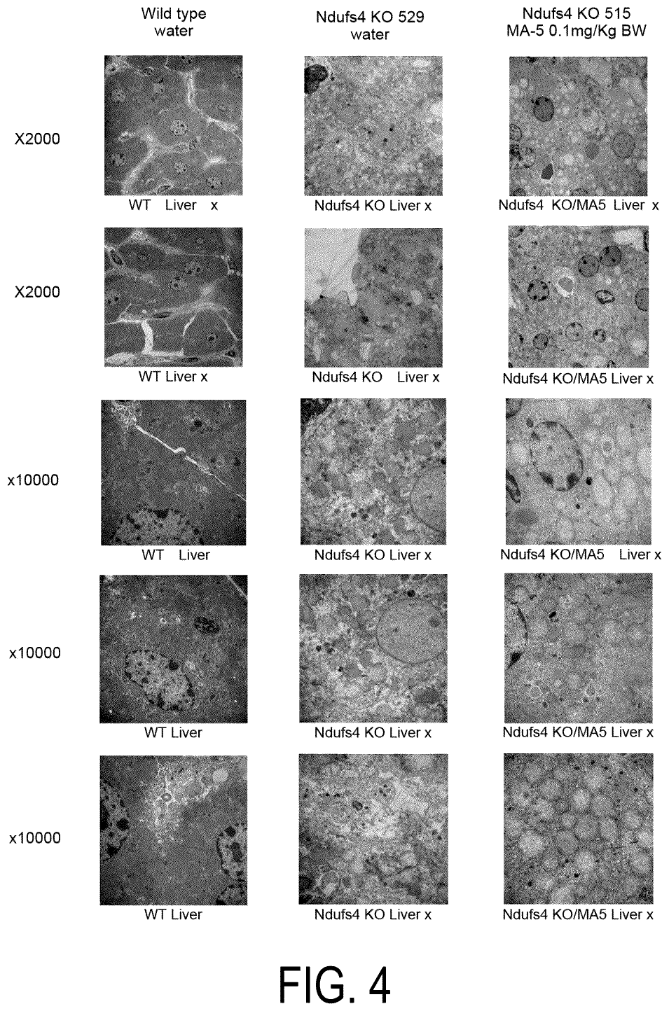

FIG. 4 shows the results of electron microscopic observation of sections of liver tissue collected from the mice of [Compound #5 administration group], [Control 1 group] and [Control 2 group].

### [Results]

As can be seen from FIG. 4, in [Control 1 group], there was a finding of fatty liver in which marked fat deposition was observed in hepatocytes, whereas in [Compound #5 administration group], there was a finding of clear reduction of fat deposition in hepatocytes and amelioration of fatty liver. The results shown in FIG. 4 suggest that the present compound group (Compound #5) has an effect of ameliorating fatty liver caused by aging.

### 5. Confirmation of Liver Function Ameliorating Effect of Compound #4 as Present Compound

### [Method]

Compound #4 of DMSO corn oil was orally administered to C57BL6 mice (8 weeks old) at a dose of 50 mg/kg/day per kg of mouse weight for 7 days ([Group L]).

In addition, Compound #4 of DMSO corn oil was orally administered to C57BL6 mice (8 weeks old) at a dose of 150 mg/kg/day per kg of mouse weight for 7 days ([Group H]).

Further, DMSO corn oil was orally administered to C57BL6 mice (8 weeks old) for 7 days ([Control group]).

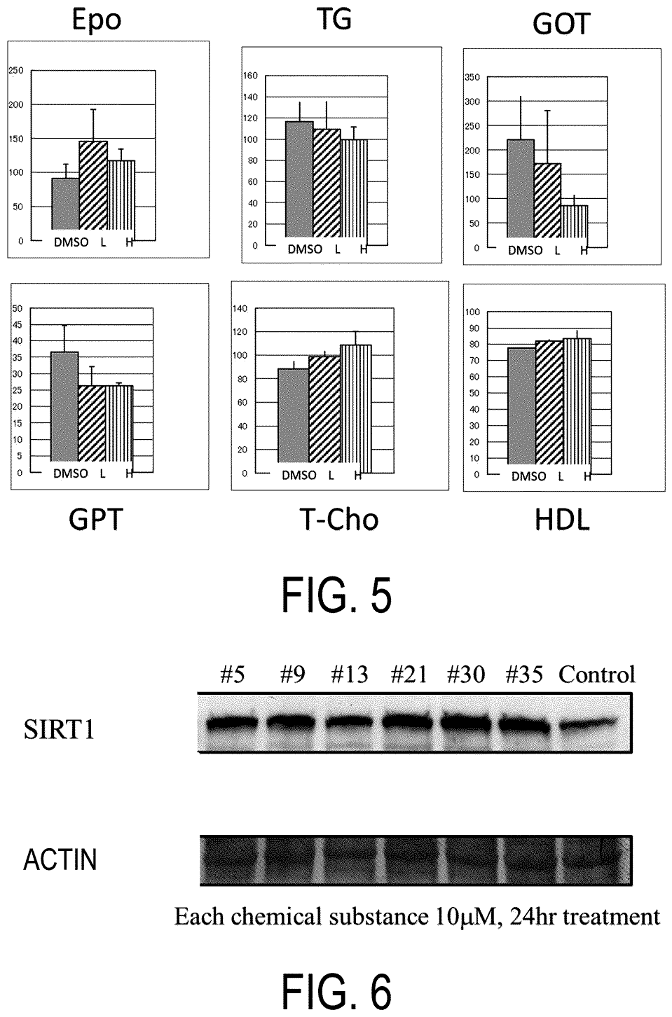

FIG. 5 shows the results of measuring the erythropoietin concentration, triglyceride concentration, GOT concentration, GTP concentration, total cholesterol (T-Cho) concentration, and HDL concentration in the blood collected from the mice of [Group L], [Group H], and [Control Group].

### [Results]

As can be seen from FIG. 5, when the present compound group (Compound #4) was administered, GOT and GPT decreased, indicating that the present compound group (Compound #4) has a liver protecting function and has an effect of ameliorating liver function.

### 6. Confirmation of SIRT Protein Production Promoting Effect of Present Compounds Other Than Compound #5

### [Method]

RH30 cells (human rhabdomyosarcoma cell line; gli gene expression+, N-myc gene expression+) at 1×10⁶ cell/dish were seeded and cultured in a dish containing 10 mL of a culture solution (low-glucose-containing DMEM medium (glucose concentration of 1.0 g/dL) to which 10 wt. % of FBS was added). One day later, Compound #5 was added to have a concentration of 30 μM, and the mixture was cultured for 24 hours ([Compound #5 addition group]). Compound #9, Compound #13, Compound #21, Compound #30, or Compound #35 as the present compound was added in place of Compound #5, and the mixture was cultured in the same manner, respectively, as [Compound #9 addition group], [Compound #13 addition group], [Compound #21 addition group], [Compound #30 addition group], and [Compound #13 addition group].

On the other hand, as a control, DMSO (dimethyl sulfoxide) was added to have a concentration of 0.2 wt. %, and the mixture was cultured for 24 hours ([Control group]).

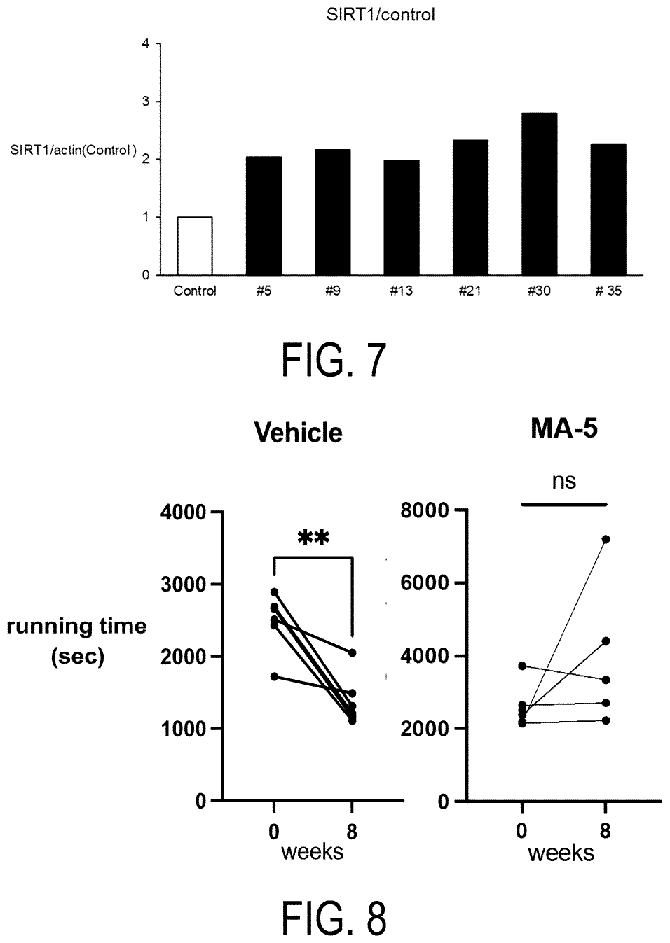

After completion of culture, the cells were recovered, disrupted by a typical method, and the amount of each of the proteins (SIRTI proteins, etc.) in the cells was detected by Western blotting. The results are shown in FIG. 6. Further, the results of quantification of each band in the detection results of the Western blotting in FIG. 6 through normalization with the band of β-actin as a loading control are shown in FIG. 7.

### [Results]

Not only in [Compound #5 addition group] but also in all of [Compound #9 addition group], [Compound #13 addition group], [Compound #21 addition group], [Compound #30 addition group], and [Compound #13 addition group], the amount of SIRTI protein in cells increased as compared with “Control group”(FIGS. 6 and 7).

These results indicate that not only Compound #5 but also many of the present compound groups have the effect of promoting the production of SIRTI proteins in cells.

### 7. Confirmation of Inhibitory Effect of Compound #5 on Decline in Motor Function of Aged Mice

### [Method]

Water-suspended Compound #5 was orally administered to C57BL6 mice (92 weeks old) at a dose of 50 mg/kg/day per kg of mouse weight for 2 months ([MA5 addition group], n=5). After 2 months of oral administration, the mice were 101 weeks old.

In addition, water was orally administered to C57BL6 mice (92 weeks old) for 2 months ([Vehicle group], n=5). After 2 months of oral administration, the mice were 101 weeks old.

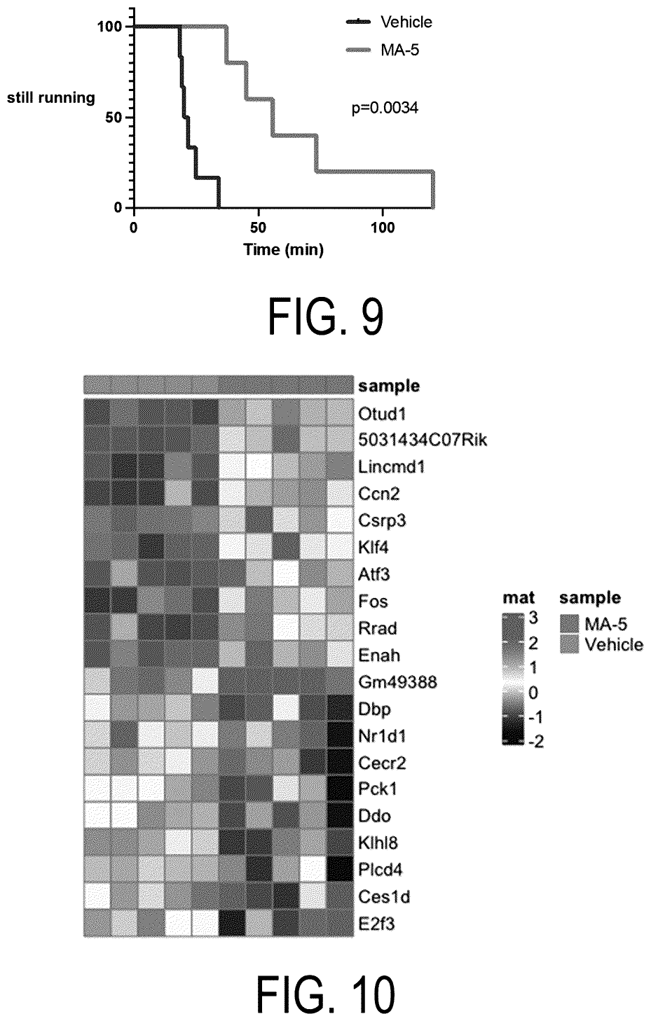

FIG. 8 shows the results of measuring the running time on a treadmill of aged mice in [MA5 addition group] and [Vehicle group]. FIG. 9 shows the results of calculation of the percentage of survival (percentage of mice running without being exhausted until the elapsed time) (%) of aged mice in [MA5 addition group] and [Vehicle group].

### [Results]

As can be seen from FIG. 8, in the aged mice of [Vehicle group], the running time on a treadmill was significantly reduced and the motor function was lowered, whereas in the aged mice of [MA5 addition group], the running time on a treadmill was not reduced and decline of the motor function was inhibited. In addition, as can be seen from FIG. 9, compared to the aged mice in [Vehicle group], the time until exhaustion was significantly improved in the aged mice in [MA5 addition group] (p =0.0034).

From these results, it was shown that Compound #5 has an inhibitory effect on the decline in motor function due to aging in aged mice.

### 8. Confirmation of Upregulated Gene Pathway and the Like in Muscle Cells of Aged Mice Treated with Compound #5

### [Method]

Water-suspended Compound #5 was orally administered to C57BL6 mice (92 weeks old) at a dose of 50 mg/kg/day per kg of mouse weight for 2 months ([MA5 addition group], n=5). After 2 months of oral administration, the mice were 101 weeks old.

In addition, water was orally administered to C57BL6 mice (92 weeks old) for 2 months ([Vehicle group], n=5). After 2 months of oral administration, the mice were 101 weeks old.

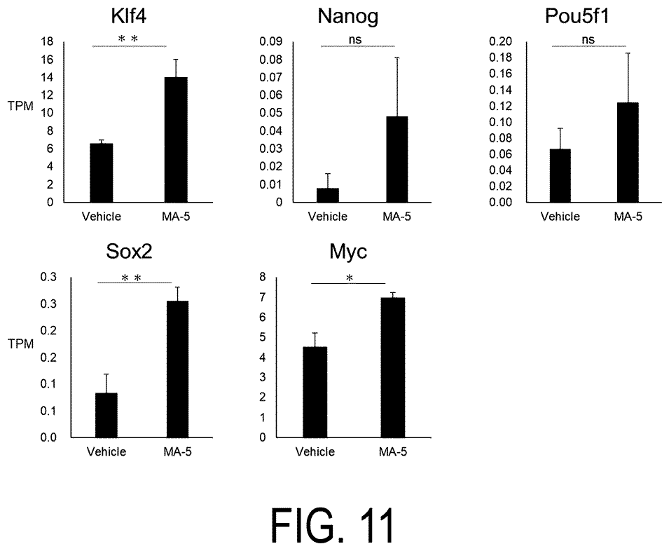

RNA sequencing (RNA-Seq) analysis was performed on the muscle cells of aged mice in [MA5 addition group] and [Vehicle group] to identify genes whose expression levels changed (increased or decreased) in [MA5 addition group] and [Vehicle group] compared to the muscle cells of young mice before administration. The results are shown in FIG. 10. FIG. 11 shows the results of comparing the expression levels of Klf4 Gene, Nanog Gene, Pou5f1 (Oct4) Gene, Sox2 Gene, and Myc Gene, which are pluripotency-related genes, between [Vehicle group] and [MA5 addition group] of aged mice.

### [Results]

In FIG. 10, the results in the five columns on the right are the results of [MA5 addition group], and the results in the five columns on the left are the results of [Vehicle group]. In FIG. 10, ten types of genes from the top are groups of genes in which an increase tendency of the expression level was observed in [MA5 addition group] and genes in which a decrease tendency of the expression level was observed in [Vehicle group], compared to before administration, and ten types of genes from the bottom are groups of genes in which a decrease tendency of the expression level was observed in [MA5 addition group] and genes in which an increase tendency of the expression level was observed in [Vehicle group], compared to before administration. As can be seen from FIG. 11, in the muscle cells of the aged mice in [MA5 addition group], the expression levels of Klf4 Gene, Sox2 Gene, and Myc Gene, which are pluripotency-related genes, were significantly increased, and the expression levels of Nanog Gene and Pou5f1 (Oct4) Gene tended to increase.

### [Method]

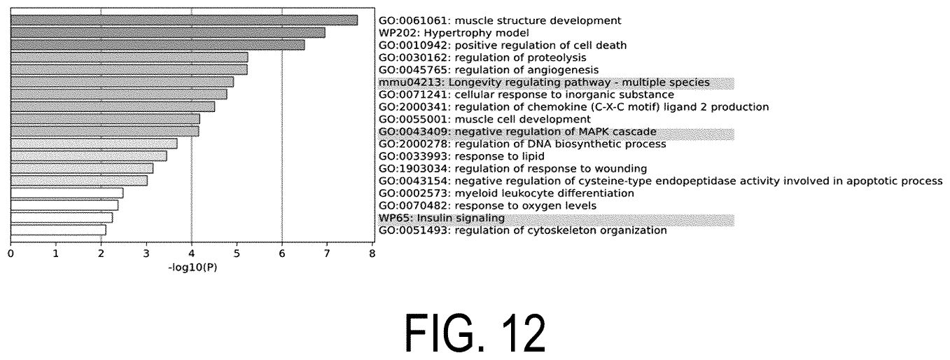

Based on the names of genes whose expression levels increased or decreased in the aged mice of [MA5 addition group] as compared with the aged mice of [Vehicle group] in the RNA sequence (RNA-Seq) analysis, pathway analysis of genes whose expression levels varied in muscle cells of the aged mice of [MA5 addition group] was performed. The results are shown in FIG. 12.

### [Results]

As can be seen from FIG. 12, it was shown that enhancement of the longevity regulating pathway, inhibition of the MAPK cascade, enhancement of the insulin signaling pathway, and the like occurred in the aged mice in [MA5 addition group].

### [Method]

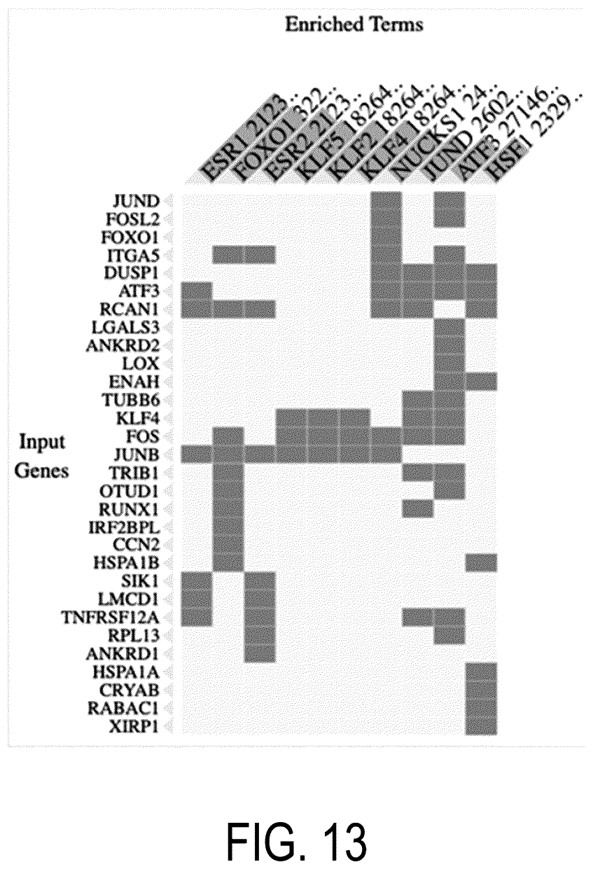

The results of the names of the genes whose expression levels increased or decreased in the aged mice of [MA5 addition group] as compared with the aged mice of [Vehicle group] in the RNA sequence (RNA-Seq) analysis were used to perform integrated analysis with ChIP-Atlas to investigate transcription factors possibly related to these genes. The results are shown in Table 1 and FIG. 13.

### [Results]

TABLE 1

| | | | Adjusted | Odds | Combined |
|---|---|---|---|---|---|
| Index | Name | P-value | p-value | Ratio | score |
| 1 | ESR1 21235772 ChIP-Seq MCF-7 Human | 0.00001149 | 0.001157 | 13.58 | 154.43 |
| 2 | FOXO1 32281255 ChIP-Seq Chondrocytes | 7.682e−7 | 0.0005109 | 9.10 | 128.05 |
| | Human Osteoarthritis | | | | |
| 3 | ESR2 21235772 ChIP-Seq MCF-7 Human | 0.000004045 | 0.0006724 | 10.07 | 125.01 |
| 4 | KLF5 18264089 ChIP-ChIP MESCs Mouse | 0.001332 | 0.02460 | 15.14 | 100.26 |
| 5 | KLF2 18264089 ChIP-ChIP MESCs Mouse | 0.001332 | 0.02460 | 15.14 | 100.26 |
| 6 | KLF4 18264089 ChIP-ChIP MESCs Mouse | 0.001332 | 0.02460 | 15.14 | 100.26 |
| 7 | NUCKS1 24931609 ChIP-Seq HEPATOCYTES | 0.00001218 | 0.001157 | 7.41 | 83.80 |
| | Mouse | | | | |
| 8 | JUND 26020271 ChIP-Seq HCASMC Human | 0.00002655 | 0.001882 | 6.68 | 70.39 |
| | CoronaryArtery | | | | |
| 9 | ATF3 27146783 Chip-Seq COLON Human | 0.000001591 | 0.0005291 | 5.20 | 69.43 |
| 10 | HSF1 23293686 ChIP-Seq STHDH STRIATAL | 0.00003392 | 0.002051 | 6.47 | 66.54 |
| | Mouse | | | | |

As can be seen from Table 1 and FIG. 13, it was shown that the transcription factors associated with genes whose expression was increased in the aged mice of [MA5 addition group] include FOXO1 known as longevity genes, KLF4, which is one of reprogramming factors necessary for the production of iPS cells, and KLF2 and KLF5, which are pluripotency-related genes.

### 9. Confirmation of Inhibitory Effect of Compound #5 on Decline in Motor Function of Young Mice

### [Method]

Water-suspended Compound #5 was orally administered to C57BL6 mice (12 weeks old) at a dose of 50 mg/kg/day per kg of mouse weight for 2 months ([MA5 high dose addition group], n=5).

Water-suspended Compound #5 was orally administered to C57BL6 mice (12 weeks old) at a dose of 10 mg/kg/day per kg of mouse weight for 2 months ([MA5 low dose addition group], n=5).

In addition, water was orally administered to C57BL6 mice (12 weeks old) for 2 months ([Vehicle group], n=5).

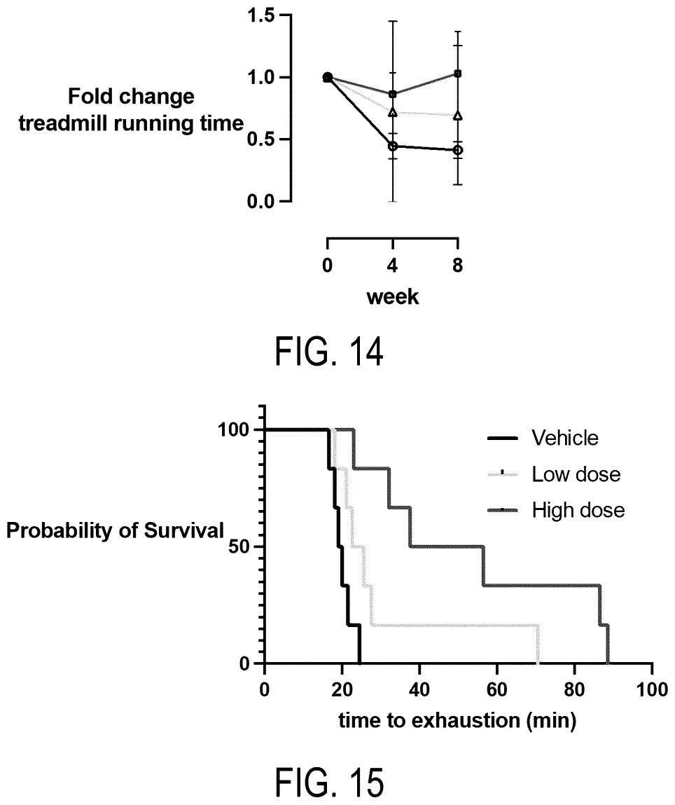

FIG. 14 shows the results of measuring the running time on a treadmill of the young mice in each of [MA5 high dose addition group], [MA5 low dose addition group], and [Vehicle group]. FIG. 15 shows the results of calculation of the percentage of survival (percentage of mice running without being exhausted until the elapsed time) (%) of the young mice in each of [MA5 high dose addition group], [MA5 low dose addition group], and [Vehicle group].

### [Results]

As can be seen from FIG. 14, in the young mice of [Vehicle group], the running time on a treadmill decreased and the motor function declined, whereas in the young mice of [MA5 low dose addition group] and [MA5 high dose addition group], the decrease in the running time on a treadmill was inhibited in a MA5 dose-dependent manner and the decline in the motor function was inhibited. In addition, as can be seen from FIG. 15, compared to the young mice in [Vehicle group], the time to exhaustion was significantly improved in the young mice in [MA5 low dose addition group] and [MA5 high dose addition group] (p<0.01).

From these results, it was shown that Compound #5 has an inhibitory effect on the decline in motor function due to aging in young mice, too.

### 10. Confirmation of Upregulated Gene Pathway and the Like in Muscle Cells of Young Mice Treated with Compound #5

### [Method]

Water-suspended Compound #5 was orally administered to C57BL6 mice (12 weeks old) at a dose of 50 mg/kg/day per kg of mouse weight for 2 months ([MA5 addition group], n=5). After 2 months of oral administration, the mice were 20 weeks old.

In addition, water was orally administered to C57BL6 mice (12 weeks old) for 2 months ([Vehicle group], n=5). After 2 months of oral administration, the mice were 20 weeks old.

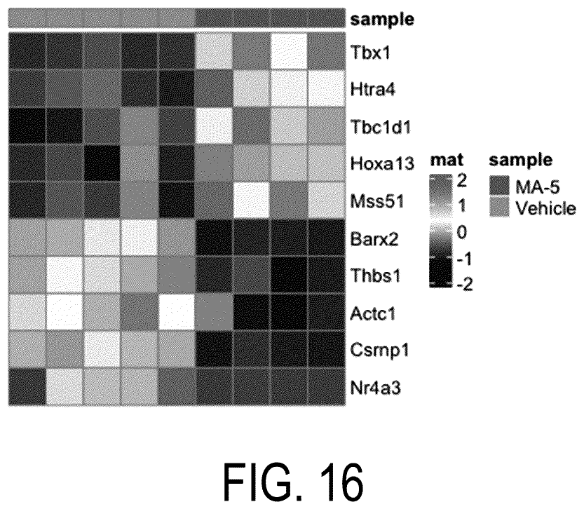

RNA sequencing (RNA-Seq) analysis was performed on the muscle cells of the young mice in [MA5 addition group] and [Vehicle group] to identify genes whose expression levels changed (increased or decreased) in [MA5 addition group] and [Vehicle group] compared to the muscle cells of young mice before administration. The results are illustrated in FIG. 16.

### [Results]

In FIG. 16, the results in the five columns on the right are the results of [MA5 addition group], and the results in the five columns on the left are the results of [Vehicle group]. In FIG. 16, five types of genes from the top are groups of genes in which an increase tendency of the expression level was observed in [MA5 addition group] and genes in which a decrease tendency of the expression level was observed in [Vehicle group], compared to before administration, and five types of genes from the bottom are groups of genes in which a decrease tendency of the expression level was observed in [MA5 addition group] and genes in which an increase tendency of the expression level was observed in [Vehicle group], compared to before administration. As can be seen from FIG. 16, in the young mice of [MA5 addition group], the expression levels of genes related to differentiation, development, and the like of muscles such as Tbx1 Gene increased.

### [Results]

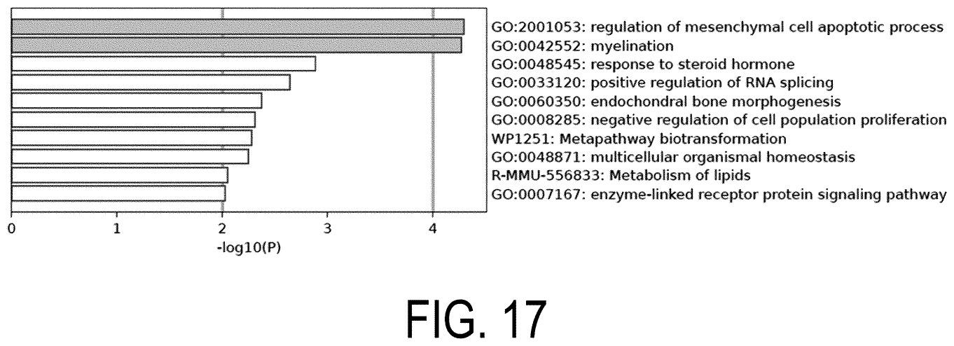

Based on the names of genes whose expression levels increased or decreased in the young mice of [MA5 addition group] as compared with the young mice of [Vehicle group] in the RNA sequence (RNA-Seq) analysis, pathway analysis of genes whose expression levels varied in [MA5 addition group] was performed. The results are illustrated in FIG. 17.

### [Results]

As can be seen from FIG. 17, in the young mice of [MA5 addition group], it was shown that genetic pathways different from those of the aged mice of [MA5 addition group] (FIG. 12) were enhanced.

### [Method]

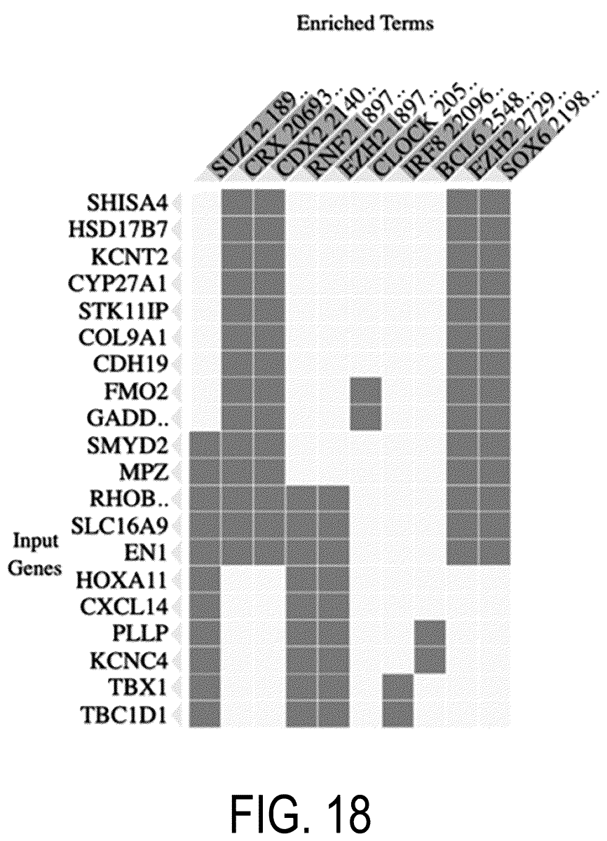

The results of the names of the genes whose expression levels increased or decreased in the young mice of [MA5 addition group] as compared with the young mice of [Vehicle group] in the RNA sequence (RNA-Seq) analysis were used to perform integrated analysis with ChIP-Atlas to investigate transcription factors possibly related to these genes. The results are shown in Table 2 and FIG. 18.

### [Results]

TABLE 2

| | | | Adjusted | Odds | Combined |
|---|---|---|---|---|---|
| Index | Name | P-value | p-value | Ratio | score |
| 1 | SUZ12 18974828 ChIP-Seq MESCs Mouse | 0.0001094 | 0.05586 | 2.73 | 24.86 |
| 2 | CRX 20693478 ChIP-Seq RETINA Mouse | 0.0001594 | 0.05586 | 2.71 | 23.69 |
| 3 | CDX2 21402776 ChIP-Seq INTESTINAL-VILLUS | 0.0005303 | 0.1239 | 2.51 | 18.94 |
| | Mouse | | | | |
| 4 | RNF2 18974828 ChIP-Seq MESCs Mouse | 0.001517 | 0.1971 | 2.61 | 16.94 |
| 5 | EZH2 18974828 ChIP-Seq MESCs Mouse | 0.001517 | 0.1971 | 2.61 | 16.94 |
| 6 | CLOCK 20551151 ChIP-Seq 293T Human | 0.001942 | 0.1971 | 3.79 | 23.69 |
| 7 | IRF8 22096565 ChIP-ChIP GC-B Human | 0.001968 | 0.1971 | 6.02 | 37.53 |
| 8 | 8CL6 25482012 ChIP-Seq CML-JURL-MK1 Human | 0.002654 | 0.2153 | 2.21 | 13.13 |
| 9 | EZH2 27294783 Chip-Seq NPCs Mouse | 0.003052 | 0.2153 | 2.49 | 14.45 |
| 10 | SOX6 21985497 ChIP-Seq MYOTUBES Mouse | 0.003083 | 0.2153 | 2.23 | 12.88 |

As can be seen from Table 2 and FIG. 18, the transcription factors related to the genes whose expression increased in the muscle cells of the young mice of [MA5 addition group] included SUZ¹² and EZH2, which are factors constituting the polycomb repressive complex-2 (PRC2) that represses histone methylations.

### 11. Confirmation of Upregulated Gene Pathways in Liver Cells of Young Mice Treated with Compound #5

### [Method]

Water-suspended Compound #5 was orally administered to C57BL6 mice (12 weeks old) at a dose of 50 mg/kg/day per kg of mouse weight for 2 months ([MA5 addition group], n=5). After 2 months of oral administration, the mice were 20 weeks old.

In addition, water was orally administered to C57BL6 mice (12 weeks old) for 2 months ([Vehicle group], n=5). After 2 months of oral administration, the mice were 20 weeks old.

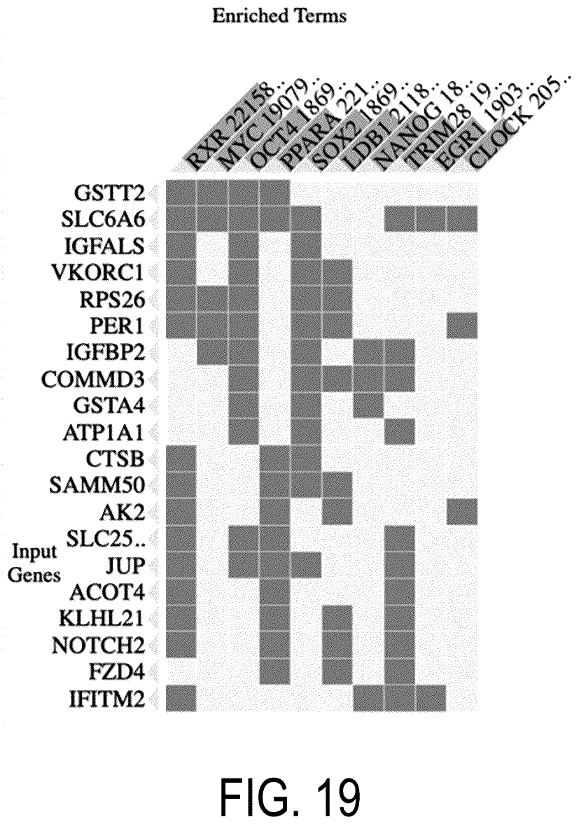

RNA sequencing (RNA-Seq) analysis was performed on the liver cells of young mice in [MA5 addition group] and [Vehicle group] to identify genes whose expression levels changed (increased or decreased) in [MA5 addition group] as compared with [Vehicle group]. Using the results of these gene names, integrated analysis with ChIP-Atlas was performed to investigate transcription factors possibly related to these genes. The results are shown in Table 3 and FIG. 19.

### [Results]

TABLE 3

| | | | Adjusted | Odds | Combined |
|---|---|---|---|---|---|
| Index | Name | P-value | p-value | Radio | score |
| 1 | RXR 22158963 ChIP-Seq LIVER Mouse | 0.00001432 | 0.009824 | 3.58 | 39.88 |
| 2 | MYC 19079543 ChIP-ChIP MESCs Mouse | 0.0002613 | 0.02561 | 3.51 | 28.98 |
| 3 | OCT4 18692474 ChIP-Seq MEFs Mouse | 0.0001240 | 0.02561 | 3.09 | 27.82 |
| 4 | PPARA 22158963 ChIP-Seq LIVER Mouse | 0.0001821 | 0.02561 | 3.08 | 26.56 |
| 5 | SOX2 18692474 ChIP-Seq MEFs Mouse | 0.0002028 | 0.02561 | 3.05 | 25.97 |
| 6 | LDB1 21186366 ChIP-Seq BM-HSCs Mouse | 0.0002384 | 0.02561 | 3.11 | 25.97 |
| 7 | RBPJ 22232070 ChIP-Seq NCS Mouse | 0.09504 | 0.5433 | 10.55 | 24.82 |
| 8 | NANOG 18700969 ChIP-ChIP MESCs Mouse | 0.009004 | 0.2376 | 5.20 | 24.50 |
| 9 | TRIM28 19339689 ChIP-ChIP MESCs Mouse | 0.0002495 | 0.02561 | 2.70 | 22.40 |
| 10 | EGR1 19032775 ChIP-ChIP M12 Human | 0.01138 | 0.2603 | 4.84 | 21.68 |

As can be seen from Table 3 and FIG. 19, transcription factors related to genes whose expression increased in liver cells of young mice in [MA5 addition group] include MYC, OCT4, SOX2, and NANOG related to reprogramming factors in the production of iPS cells, RXR (retinoid X receptor) and PPARA (peroxisome proliferator-activated receptor a) related to SIRT, and TRIM28 (tripartite motif-containing proteins 28) related to histone methylations.

### 12. Confirmation of Upregulated Gene Pathway and the Like in Liver Cells of Aged Mice Treated with Compound #5

### [Method]

Water-suspended Compound #5 was orally administered to C57BL6 mice (92 weeks old) at a dose of 50 mg/kg/day per kg of mouse weight for 2 months ([MA5 addition group], n=5). After 2 months of oral administration, the mice were 101 weeks old.

In addition, water was orally administered to C57BL6 mice (92 weeks old) for 2 months ([Vehicle group], n=5). After 2 months of oral administration, the mice were 101 weeks old.

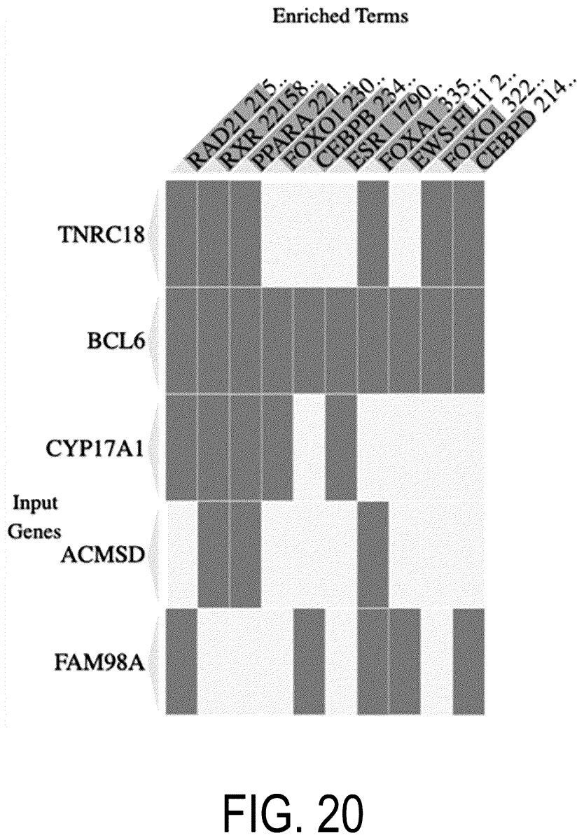

RNA sequencing (RNA-Seq) analysis was performed on the liver cells of aged mice in [MA5 addition group] and [Vehicle group] to identify genes whose expression levels changed (increased or decreased) in [MA5 addition group] as compared with the liver cells of young mice before administration. Using the results of these gene names, integrated analysis with ChIP-Atlas was performed to investigate transcription factors possibly related to these genes. The results are shown in Table 4 and FIG. 20.

### [Results]

TABLE 4

| | | | Adjusted | Odds | Combined |
|---|---|---|---|---|---|
| Index | Name | P-value | p-value | Ratio | score |
| 1 | RAD21 21589869 ChIP-Seq MESCs Mouse | 0.01064 | 0.7446 | 5.98 | 27.18 |
| 2 | RXR 22158963 ChIP-Seq LIVER Mouse | 0.01064 | 0.7446 | 5.98 | 27.18 |
| 3 | PPARA 22158963 ChIP-Seq LIVER Mouse | 0.01078 | 0.7446 | 5.96 | 26.98 |
| 4 | FOXO1 23066095 ChIP-Seq LIVER Mouse | 0.01143 | 0.7446 | 14.39 | 64.34 |
| 5 | CEBPB 23403033 ChIP-Seq LIVER Mouse | 0.01374 | 0.7446 | 13.04 | 55.89 |
| 6 | ESR1 17901129 ChIP-ChIP LIVER Mouse | 0.01834 | 0.7446 | 11.16 | 44.61 |
| 7 | FOXA1 33576154 ChIP-Seq Human Liver | 0.02328 | 0.7446 | 4.65 | 17.49 |
| | ProstateCancer | | | | |
| 8 | EWS-FLI1 20517297 ChIP-Seq SK-N-MC Human | 0.03112 | 0.7446 | 8.34 | 28.94 |
| 9 | FOXO1 32281255 ChIP-Seq Chondrocytes Human | 0.03894 | 0.7446 | 7.36 | 23.88 |
| | Osteoarthritis | | | | |
| 10 | CEBPD 21427703 ChIP-Seq 3T3-L1 Mouse | 0.04256 | 0.7446 | 4.62 | 14.57 |

As can be seen from Table 4 and FIG. 20, it was shown that transcription factors related to genes whose expression increased in the liver cells of aged mice in [MA5 addition group] include FOXO1 known as longevity genes and PPARA (peroxisome proliferator-activated receptor α) related to SIRT.

### 13. Confirmation of Genetic Pathway and the Like Upregulated in Cochlear Cells of Ndufs4 Knockout Mice Treated with Compound #5

### [Method]

Water-suspended Compound #5 was orally administered to Ndufs4 (NADH-ubiquinone oxidoreductase subunit 4) knockout mice (6 weeks old) at a dose of 50 mg/kg/day per kg of mouse weight for 2 weeks ([MA5 addition group], n=5).

In addition, water was orally administered to Ndufs4 knockout mice (6 weeks old) for 2 weeks ([Vehicle group], n=5).

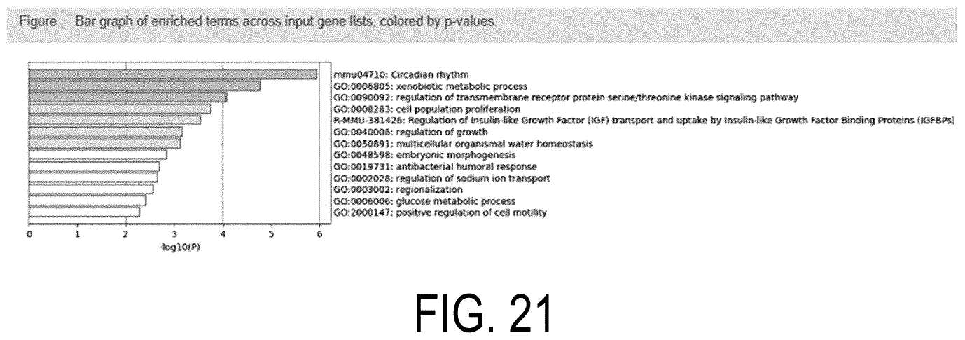

RNA sequence (RNA-Seq) analysis was performed on cochlear cells of the inner ear of Ndufs4 knockout mice of [MA5 addition group] and [Vehicle group] described above, and genes whose expression levels changed (increased or decreased) in [MA5 addition group] as compared with [Vehicle group] were identified. Based on the names of the genes, pathway analysis was performed on genes whose expression has varied in the inner ear cells of the Ndufs4 knockout mice in [MA5 addition group]. The results are shown in FIG. 21.

### [Results]

As can be seen from FIG. 21, in the cochlear cells of the inner ears of the Ndufs4 knockout mice of [MA5 addition group], the pathway of the insulin like growth factor (IGF) genes was enhanced, indicating that the IGF1 signaling pathway was enhanced by the MA5 treatment

### [Method]

The results of the names of the genes whose expression levels increased or decreased in the Ndufs4 knockout mice of [MA5 addition group] as compared with the Ndufs4 knockout mice of [Vehicle group] in the RNA sequence (RNA-Seq) analysis were used to perform integrated analysis with ChIP-Atlas to investigate transcription factors possibly related to these genes. The results are shown in Table 5.

### [Results]

TABLE 5

| | | | Adjusted | Odds | Combined |
|---|---|---|---|---|---|
| Index | Name | P-value | p-value | Ratio | score |
| 1 | RUNX2 24655370 ChIP-Seq MC3T3E1 Mouse | 0.00003554 | 0.01485 | 4.69 | 48.01 |
| | Bone | | | | |
| 2 | SOX2 30713093 ChIP-Seq Epithelial Human | 0.0004025 | 0.08412 | 6.18 | 48.28 |
| | Tongue SCC | | | | |
| 3 | POU2F1 27270436 ChIP-Seq LNCaP Human | 0.0007988 | 0.1113 | 7.90 | 56.36 |
| | Prostate Carcinoma | | | | |
| 4 | POU2F1 27270436 ChIP-Seq VCaP Human | 0.002300 | 0.2403 | 5.14 | 31.20 |
| | Prostate Carcinoma | | | | |
| 5 | PITX1 30713093 ChIP-Seq Epithelial Human | 0.01159 | 0.9489 | 13.26 | 59.11 |
| | Tongue SCC | | | | |
| 6 | MYC 30127528 ChIP-Seq KELLY Human Brain | 0.01362 | 0.9489 | 2.52 | 10.83 |
| | Neuroblastoma | | | | |
| 7 | TBX2 30127528 ChIP-Seq KELLY Human Brain | 0.02324 | 0.9972 | 2.64 | 9.93 |
| | Neuroblastoma | | | | |
| 8 | FOXM1 26456572 ChIP-Seq MCF-7 Human | 0.03417 | 0.9972 | 4.56 | 15.40 |
| | BreastCancer | | | | |
| 9 | STAT5B 24692510 ChIP-Seq Epithelium Mouse | 0.03526 | 0.9972 | 4.50 | 15.06 |
| | Mammary | | | | |
| 10 | FOXM1 23109430 ChIP-Seq U2OS Human | 0.04495 | 0.9972 | 6.30 | 19.53 |

As can be seen from Table 5, it was shown that transcription factors related to genes whose expression increased in the cells of the cochlea of the inner ears of Ndufs4 knockout mice of the [MA5 addition group] include SOX2 and MYC, which are reprogramming factors necessary for the production of iPS cells, and POU2F1 (OCT1), which is a pluripotency-related gene.

### 14. Confirmation of Genetic Pathway and the Like Upregulated in Cochlear Cells of Wild-Type Mice Treated with Compound #5

### [Method]

Water-suspended Compound #5 was orally administered to C57BL6 mice (6 weeks old) at a dose of 50 mg/kg/day per kg of mouse weight for 2 weeks ([MA5 addition group], n=5).

In addition, water was orally administered to C57BL6 mice (6 weeks old) for 2 months ([Vehicle group], n=5).

RNA sequence (RNA-Seq) analysis was performed on cochlear cells of the inner ear of wild-type mice of [MA5 addition group] and [Vehicle group] described above, and genes whose expression levels changed (increased or decreased) in [MA5 addition group] as compared with [Vehicle group] were identified. Using the results of these gene names, integrated analysis with ChIP-Atlas was performed to investigate transcription factors possibly related to these genes. The results are shown in Table 6.

### [Results]

TABLE 6

| | | | Adjusted | Odds | Combined |
|---|---|---|---|---|---|
| Index | Name | P-value | p-value | Ratio | score |
| 1 | JARID2 20064375 ChIP-Seq MESCs Mouse | 0.001742 | 0.4320 | 5.49 | 34.88 |
| 2 | EZH2 27304074 Chip-Seq ESCs Mouse | 0.002979 | 0.4320 | 5.84 | 33.95 |
| 3 | RING1B 27294783 Chip-Seq ESCs Mouse | 0.006508 | 0.6291 | 3.72 | 18.71 |
| 4 | SUZ12 18692474 ChIP-Seq MEFs Mouse | 0.01256 | 0.8374 | 4.06 | 17.79 |
| 5 | JARID2 20075857 ChIP-Seq MESCs Mouse | 0.01444 | 0.8374 | 3.92 | 16.60 |
| 6 | SUZ12 27294783 Chip-Seq ESCs Mouse | 0.02476 | 0.9994 | 3.03 | 11.19 |
| 7 | SUZ12 18555785 ChIP-Seq MESCs Mouse | 0.03126 | 0.9994 | 3.69 | 12.80 |
| 8 | TPS3 20018659 ChIP-ChIP R1E Mouse | 0.03643 | 0.9984 | 3.51 | 11.63 |
| 9 | SUZ12 16625203 ChIP-ChIP MESCs Mouse | 0.04099 | 0.9994 | 3.37 | 10.78 |
| 10 | MTF2 20144788 ChIP-Seq MESCs Mouse | 0.05124 | 0.9994 | 2.35 | 6.98 |

As can be seen from Table 6, the transcription factors related to the genes whose expression increased in the cochlear cells of the inner ears of the wild-type mice in [MA5 addition group] included SUZ¹² and EZH2, which are factors constituting polycomb repressive complex-2 (PRC2), which methylates histones.

### 17. Confirmation of Upregulated Genetic Pathway and the Like in Mouse Inner Ear Cells HEI-OC1 Treated with Compound #5

### [Method]

Mouse inner ear cells HEI-OC1 were cultured in 10% FBS-containing DMEM medium containing 10 UM of Compound #5 (dissolved in 0.1% DMSO) ([MA5 addition group], n=5).

In addition, mouse inner ear cells HEI-OC1 were cultured in 10% FBS-containing DMEM medium containing Compound #5 in 0.1% DMSO ([Vehicle group], n=5).

RNA sequencing (RNA-Seq) analysis was performed on the HEI-OC1 cells in [MA5 addition group] and [Vehicle group] to identify genes whose expression levels changed (increased or decreased) in [MA5 addition group] as compared with [Vehicle group]. Using the results of these gene names, integrated analysis with ChIP-Atlas was performed to investigate transcription factors possibly related to these genes. The results of ChIP enrichment analysis (ChEA) are shown in Table 7, and the results of analysis by ENCODE and ChEA consensus TFs from ChIP-X are shown in Table 8.

### [Results]

TABLE 7

| | | | Adjusted | Odds | Combined |
|---|---|---|---|---|---|
| Index | Name | P-value | p-value | Ratio | score |
| 1 | FLI1 27457419 Chip-Seq LIVER | 1.920e−17 | 1.261e−14 | 14.82 | 570.26 |
| | Mouse | | | | |
| 2 | PCGF2 27294783 Chip-Seq ESCs | 5.479e−15 | 1.800e−12 | 11.05 | 362.84 |
| | Mouse | | | | |
| 3 | SUZ12 27294783 Chip-Seq NPCs | 3.417e−11 | 7.484e−9 | 6.57 | 158.41 |
| | Mouse | | | | |
| 4 | NFE2 27457419 Chip-Seq LIVER | 3.035e−10 | 4.984e−8 | 5.84 | 127.96 |
| | Mouse | | | | |
| 5 | EZH2 27294783 Chip-Seq NPCs | 4.016e−8 | 0.000005277 | 4.28 | 72.84 |
| | Mouse | | | | |
| 6 | STAT3 18555785 Chip-Seq ESCs | 0.000002814 | 0.0002496 | 3.16 | 40.41 |
| | Mouse | | | | |
| 7 | EBF1 22473956 ChIP-Seq | 0.000003179 | 0.0002496 | 3.14 | 39.74 |
| | LYMPHODE Mouse | | | | |
| 8 | SOX2 18555785 Chip-Seq ESCs | 0.000003297 | 0.0002496 | 3.13 | 39.54 |
| | Mouse | | | | |
| 9 | P53 22387025 ChIP-Seq ESCs Mouse | 0.000003419 | 0.0002496 | 3.13 | 39.34 |
| 10 | RUNX1 27457419 Chip-Seq LIVER | 0.000007118 | 0.0003691 | 3.05 | 36.20 |
| | Mouse | | | | |

TABLE 8

| | | | Adjusted | Odds | Combined |
|---|---|---|---|---|---|
| Index | Name | P-value | p-value | Ratio | score |
| 1 | PPARG CHEA | 0.02274 | 0.9404 | 2.43 | 9.18 |
| 2 | TAF7 ENCODE | 0.02294 | 0.9404 | 2.29 | 8.63 |
| 3 | NELFE ENCODE | 0.06509 | 0.9999 | 2.73 | 7.47 |
| 4 | TAF1 ENCODE | 0.08384 | 0.9999 | 1.38 | 3.43 |
| 5 | NRF1 ENCODE | 0.1543 | 0.9999 | 1.37 | 2.55 |
| 6 | MYC CHEA | 0.1660 | 0.9999 | 1.66 | 2.98 |
| 7 | YY1 CHEA | 0.1673 | 0.9999 | 1.91 | 3.41 |
| 8 | TRIM28 ENCODE | 0.1814 | 0.9999 | 2.64 | 4.50 |
| 9 | IRF1 ENCODE | 0.2410 | 0.9999 | 1.80 | 2.56 |
| 10 | FLI1 ENCODE | 0.2430 | 0.9999 | 1.46 | 2.06 |

As can be seen from Table 7, it was shown that transcription factors related to genes whose expression increased in the HEI-OC1 cells of [MA5 addition group] include SUZ¹² and EZH2, which are factors constituting polycomb repressive complex-2 (PRC2), which methylates histones, and SOX2, which is one of reprogramming factors necessary for the production of iPS cells. In addition, as can be seen from Table 8, it was shown that PPARG (peroxisome proliferator-activated receptor γ), which is a longevity-related gene, MYC, which is one of the reprogramming factors necessary for the production of iPS cells, TRIM28 (tripartite motif-containing proteins 28), which is related to histone methylations, and the like were contained.

### 18. Confirmation of DNA Methylation Inhibitory Effect of Compound #5 in Liver Cells of Young Mice and Aged Mice

### [Method]

Water-suspended Compound #5 was orally administered to C57BL6 mice (12 weeks old) at a dose of 50 mg/kg/day per kg of mouse weight for 2 months ([MA5 high dose addition group], n=5). The degree of methylation of the genomic DNA of the liver cells of the young mice was measured by the bisulfite method. The results are shown in Table 9.

Water-suspended Compound #5 was orally administered to C57BL6 mice (92 weeks old) at a dose of 50 mg/kg/day per kg of mouse weight for 2 months ([MA5 addition group], n=5). The degree of methylation of the genomic DNA of the liver cells of the aged mice was measured by the bisulfite method. The results are shown in Table 10. In Table 9 and Table 10, “Count” represents how many genes were related among genes related to the pathway. For example, in a pathway involving 20 genes, when there are three genes whose expression levels have been varied by administration of Compound #5, Count is 3, and “ %” thereof is a numerical value calculated by (3/20)×100.

“P-value” represents the p-value of the between-group comparison of pathways, and “Benjamini” represents the p-value calculated using the linear step-up method of Benjamini and Hochberg (1995) (see, for example, https://david.ncifcrf.gov/helps/functional_annotation.html for calculation method).

From Table 9, it was shown that in the young mice of [MA5 addition group], methylations of Wnt signaling pathway, Signaling pathways regulating pluripotency of stem cells, and Insulin secretion, which are pathways related to aging, were inhibited. In addition, from Table 10, it was shown that in the aged mice of [MA5 addition group], methylations of insulin resistance and insulin signaling pathways, which are pathways related to aging, were inhibited. From these results, it was shown that Compound #5 has an effect of inhibiting the methylation of DNA of aging-related pathways.

### 19. Confirmation of Survival Rate Improving Effect Through Administration of Compound #5 to α-Klotho Knockout Mice

### [Method]

Water-suspended Compound #5 was orally administered to α-Klotho gene-deficient mice (28 days old) as a premature aging animal model at a dose of 10 mg/kg mouse weight/day, the progress was observed until death, and the survival rate was evaluated from a survival curve based on the Kaplan-Meier method (Klotho homo KO mouse administration group, n=9).

In addition, water was orally administered to α-Klotho gene-deficient mice (28 days old) until death (Klotho homo KO mouse non-administered group, n=9).

### [Results]

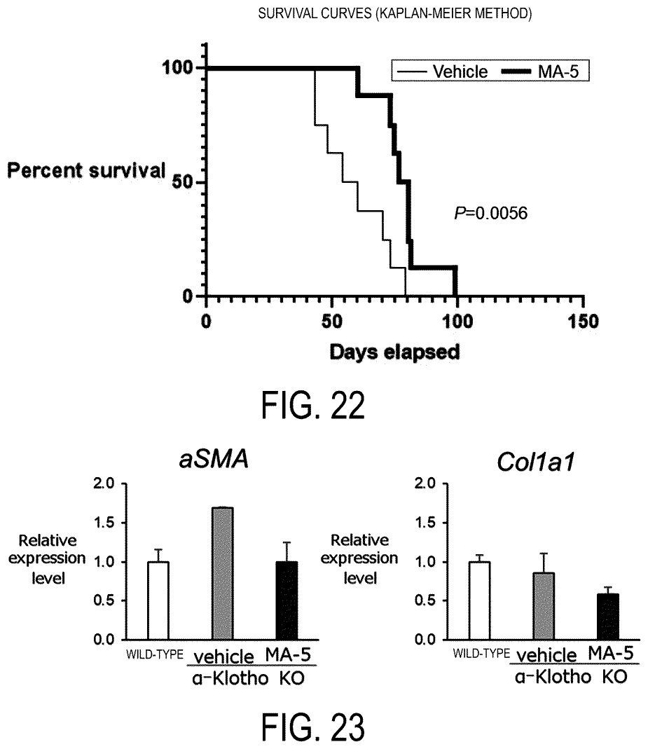

As can be seen from FIG. 22, when Compound #5 was administered to the α-Klotho gene-deficient mouse, which is a premature aging animal model, from a young age (28 days old), the survival rate was significantly increased (p =0.0056).

20. Effect of Administration of Compound #5 on Renal Fibrosis in α-Klotho Knockout Mice

### [Method]

Water-suspended Compound #5 was orally administered to α-Klotho gene-deficient mice (28 days old) at a dose of 10 mg/kg mouse weight/day until 60 days old (Klotho homo KO mouse administration group, n=3).

In addition, water was orally administered to wild-type mice (28 days old) and α-Klotho gene-deficient mice (28 days old) until 60 days old (n=3 for each of the wild-type non-administrated group and the Klotho homo KO mouse non-administrated group).

Renal tissue was collected from each 60-day-old mouse according to a standard method, total RNA was extracted using a commercially available column extraction kit (RNeasy Micro Kit, manufactured by QIAGEN), and cDNA was synthesized from 1 μg of total RNA using ReverTra Ace(manufactured by TOYOBO CO., Ltd.). Thereafter, to quantify mRNA derived from two types of fibrosis-related genes (aSMA and Colla1) and mRNA derived from GAPDH as an internal standard, real-time PCR was performed according to a standard method using QuantStudio 5 Real-time PCR system (manufactured by Applied Biosystems) as a measuring instrument and THUNDERBIRD SYBR qPCR MasterMix (manufactured by TOYOBO CO., Ltd.) as a reaction liquid.

### [Results]

As can be seen from FIG. 23, it was shown that when Compound #5 was administered to the α-Klotho gene-deficient mouse, which is a premature aging animal model, renal fibrosis progressing with aging was inhibited.

### 21. Effect on Blood Metabolites by Administration of Compound #5 to α-Klotho Knockout Mice

### [Method]

Water-suspended Compound #5 was orally administered to α-Klotho gene-deficient mice (28 days old) at a dose of 10 mg/kg mouse weight/day until 60 days old (Klotho homo KO mouse administration group, n=2).

In addition, water was orally administered to wild-type mice (28 days old) and α-Klotho gene-deficient mice (28 days old) until 60 days old (n=2 for each of the wild-type non-administrated group and the Klotho homo KO mouse non-administrated group).

Plasma was prepared from each 60-day-old mouse, and the effect of administration of Compound #5 on plasma metabolites was subjected to target analysis (comprehensive analysis) by CE-TOFMS (manufactured by Human Metabolome Technologies, Inc.).

### [Results]

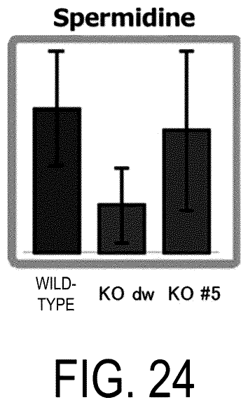

As can be seen from FIG. 24, spermidine, which is known as a longevity molecule, was identified as a plasma metabolite that increased by administration of Compound #5 to α-Klotho gene-deficient mice.

### INDUSTRIAL APPLICABILITY

According to the present invention, it is possible to provide an agent for anti-aging or cellular rejuvenation, and a preventive or ameliorative agent for symptoms or diseases associated with aging. Further, the present invention adopts a low-molecular compound that can be produced relatively simply and in a high yield as an active ingredient for anti-aging or cellular rejuvenation, and thus enables relatively simple and inexpensive production.

1. A method for anti-aging or rejuvenating cells in an animal in need of anti-aging or cellular rejuvenation, the method comprising administering to the animal one or more compounds selected from the group consisting of:
a compound represented by General Formula (I₀): 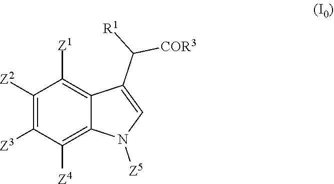
wherein
R¹ represents a hydrogen atom; a benzoylmethyl group in which a benzene ring is unsubstituted or substituted with an alkyl group having 1 to 7 carbon atoms, an alkoxyl group having 1 to 7 carbon atoms, and fluorine and/or chlorine; a chain or branched alkyl group having 4 to 6 carbon atoms, the alkyl group being unsubstituted or substituted with fluorine; or methylene or ethylene substituted with a phenyl group, a cyclopentyl group, or a piperidinyl group, wherein the phenyl group may be further substituted with one or more phenyl groups, and N of the piperidinyl group may be further substituted with an acyl group,
Z¹, Z², Z³, and Z⁴ may be identical or different, and represent a hydrogen atom, a halogen atom, a C1 to C6 alkyl group, a C2 to C6 alkenyl group, a C2 to C6 alkynyl group, or an organic oxy group represented by OR⁸, wherein R⁸ represents a C1 to C7 alkyl group, a C2 to C6 alkenyl group, a C2 to C6 alkynyl group, or a benzyl group,
Z⁵ represents a hydrogen atom or a C1 to C6 alkyl group, and
R³ is a group selected from the group consisting of OH, OR⁴, NHR⁴, and NR⁴R⁵, wherein R⁴ and R⁵ are identical or different and are a substituted or unsubstituted alkyl group having 1 to 4 carbon atoms;
a compound represented by General Formula (II): 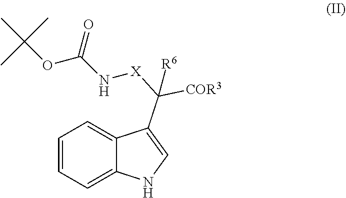
wherein
R⁶ is a hydrogen atom or a methyl group,
X is an alkylene group having 4 to 6 carbon atoms or an ether group having 4 carbon atoms, and
R³ is a group selected from the group consisting of OH, OR⁴, NHR⁴, and NR⁴R⁵, wherein R⁴ and R⁵ are identical or different and are a substituted or unsubstituted alkyl group having 1 to 4 carbon atoms;
a compound represented by General Formula (III): 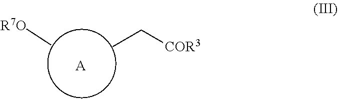
wherein
A represents indole or naphthalene,
wherein
when A is indole, an acetic acid group and R⁷O are substituted at a third position and a fifth position of indole, respectively, and
when A is naphthalene, an acetic acid group and R⁷O are substituted at a first position and a seventh position of naphthalene, respectively,
R⁷ represents an alkyl group having 1 to 5 carbon atoms or a benzyl group, wherein a benzene ring of the benzyl group may be substituted with one or more alkyl groups having 1 to 3 carbon atoms or alkoxy groups having 1 to 3 carbon atoms, and
R³ is a group selected from the group consisting of OH, OR⁴, NHR⁴, and NR⁴R⁵, wherein R⁴ and R⁵ are identical or different and are a substituted or unsubstituted alkyl group having 1 to 4 carbon atoms; and
a pharmaceutically acceptable salt thereof when R³ is OH.

2. The method according to claim 1, wherein the one or more compounds can promote the production of an SIRT protein, and wherein the SIRT protein is one or more proteins selected from SIRT1 protein, SIRT2 protein, SIRT3 protein, SIRT4 protein, SIRT5 protein, SIRT6 protein, and SIRT7 protein.

3. The method according to claim 1, wherein the compound is a compound represented by the following Formula (I-1), Formula (II-1), Formula (I-2), Formula (I-3), Formula (I-4), Formula (I-5) or Formula (III-1), or a pharmaceutically acceptable salt thereof:

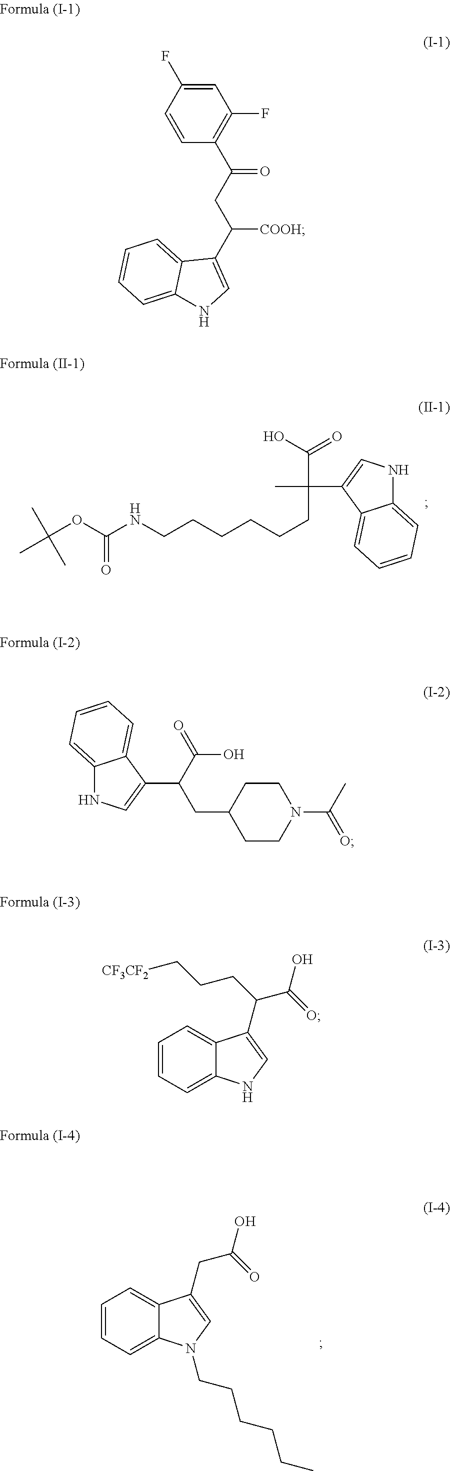

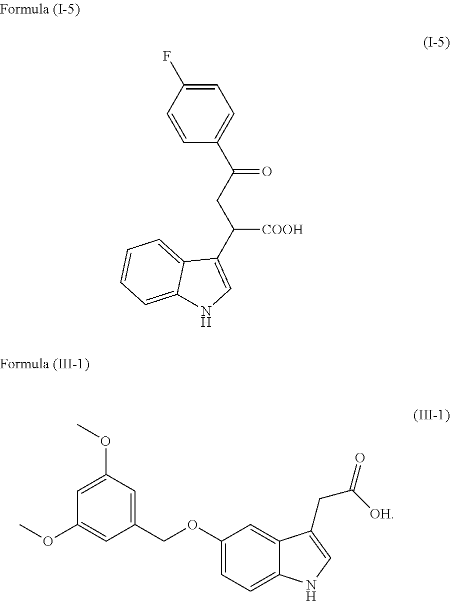 [SMILES: O=C(CC(C(=O)O)c1c[nH]c2ccccc12)c1ccc(F)cc1 ; COc1cc(COc2ccc3[nH]cc(CC(=O)O)c3c2)cc(OC)c1]

4. A method for preventing or ameliorating a symptom or disease associated with aging in a subject in need of prevention or amelioration of a symptom or disease associated with aging, the method comprising administering to the subject one or more compounds selected from the group consisting of:
a compound represented by General Formula (I₀); 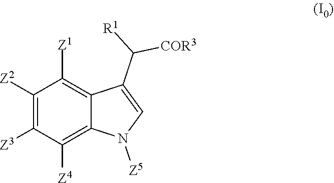
wherein
R¹ represents a hydrogen atom; a benzoylmethyl group in which a benzene ring is unsubstituted or substituted with an alkyl group having 1 to 7 carbon atoms, an alkoxyl group having 1 to 7 carbon atoms, and fluorine and/or chlorine; a chain or branched alkyl group having 4 to 6 carbon atoms, the alkyl group being unsubstituted or substituted with fluorine; or methylene or ethylene substituted with a phenyl group, a cyclopentyl group, or a piperidinyl group, wherein the phenyl group may be further substituted with one or more phenyl groups, and N of the piperidinyl group may be further substituted with an acyl group,
Z¹, Z², Z³, and Z⁴ may be identical or different, and represent a hydrogen atom, a halogen atom, a C1 to C6 alkyl group, a C2 to C6 alkenyl group, a C2 to C6 alkynyl group, or an organic oxy group represented by OR⁸ wherein R⁸ represents a C1 to C7 alkyl group, a C2 to C6 alkenyl group, a C2 to C6 alkynyl group, or a benzyl group,
Z⁵ represents a hydrogen atom or a C1 to C6 alkyl group, and
R³ is a group selected from the group consisting of OH, OR⁴, NHR⁴ and NR⁴R⁵, wherein R⁴ and R⁵ are identical or different and are a substituted or unsubstituted alkyl group having 1 to 4 carbon atoms;
a compound represented by General Formula (II): 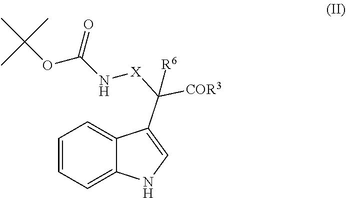
wherein
R⁶ is a hydrogen atom or a methyl group,
X is an alkylene group having 4 to 6 carbon atoms or an ether group having 4 carbon atoms, and
R³ is a group selected from the group consisting of OH, OR⁴, NHR⁴ and NR⁴R⁵, wherein R⁴ and R⁵ are identical or different and are a substituted or unsubstituted alkyl group having 1 to 4 carbon atoms;
a compound represented by General Formula (III): 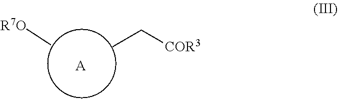
wherein
A represents indole or naphthalene,
wherein
when A is indole, an acetic acid group and R⁷O are substituted at a third position and a fifth position of indole, respectively, and
when A is naphthalene, an acetic acid group and R⁷O are substituted at a first position and a seventh position of naphthalene, respectively,
R⁷ represents an alkyl group having 1 to 5 carbon atoms or a benzyl group, wherein a benzene ring of the benzyl group may be substituted with one or more alkyl groups having 1 to 3 carbon atoms or alkoxy groups having 1 to 3 carbon atoms, and
R³ is a group selected from the group consisting of OH, OR⁴, NHR⁴ and NR⁴R⁵, wherein R⁴ and R⁵ are identical or different and are a substituted or unsubstituted alkyl group having 1 to 4 carbon atoms; and
a pharmaceutically acceptable salt thereof when R³ is OH.

5. The method according to claim 4, wherein the compound is a compound represented by the following Formula (I-1), Formula (II-1), Formula (I-2), Formula (I-3), Formula (I-4), Formula (I-5) or Formula (III-1), or a pharmaceutically acceptable salt thereof:

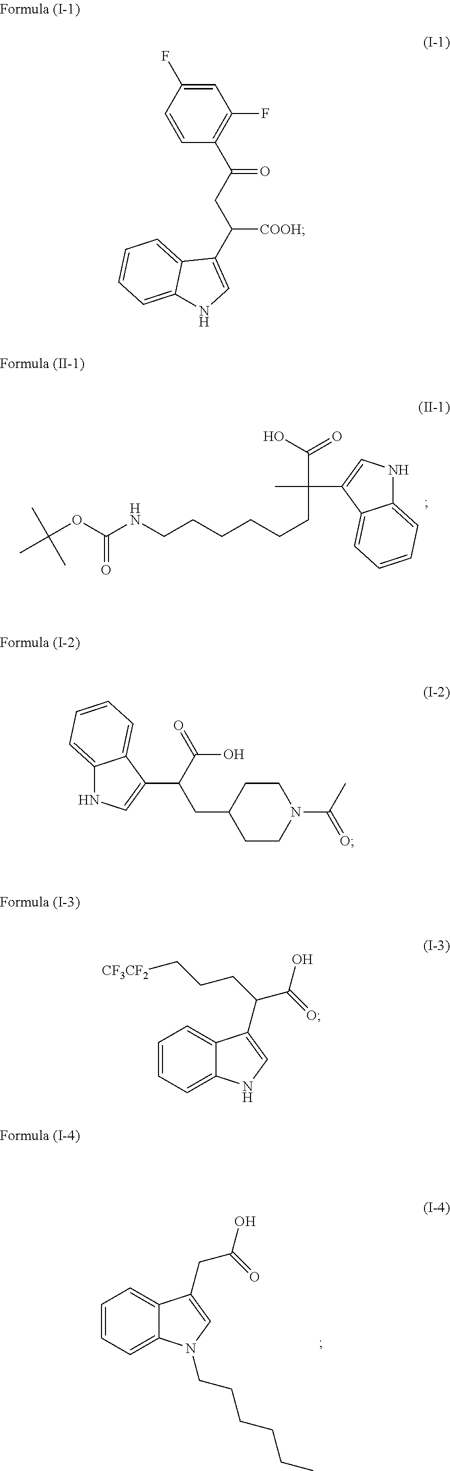

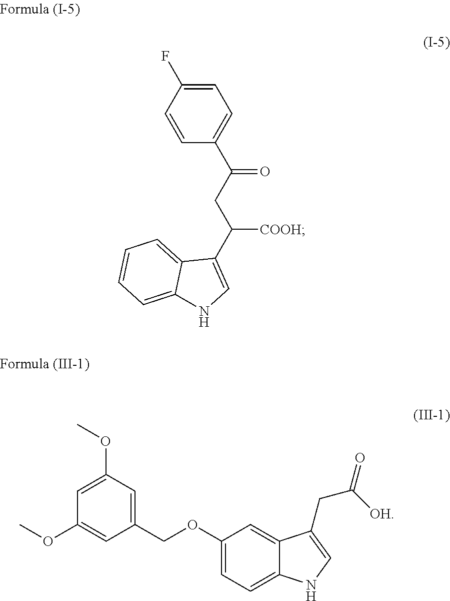 [SMILES: O=C(CC(C(=O)O)c1c[nH]c2ccccc12)c1ccc(F)cc1 ; COc1cc(COc2ccc3[nH]cc(CC(=O)O)c3c2)cc(OC)c1]

6. The method according to claim 4, wherein the symptom or disease associated with aging is one or more selected from the group consisting of obesity, liver disease, decline in liver function, renal fibrosis, renal dysfunction, and decline in motor function.

7. The method according to claim 4, wherein the one or more compounds can promote the production of an SIRT protein, and wherein the SIRT protein is one or more proteins selected from SIRT1 protein, SIRT2 protein, SIRT3 protein, SIRT4 protein, SIRT5 protein, SIRT6 protein, and SIRT7 protein.

8. The method according to claim 7, wherein the symptom or disease associated with aging is one or more selected from the group consisting of obesity, liver disease, decline in liver function, renal fibrosis, renal dysfunction, and decline in motor function.

9. The method according to claim 6, wherein the symptom or disease associated with aging is one or more selected from the group consisting of obesity, liver disease, decline in liver function, renal fibrosis, renal dysfunction, and decline in motor function.
